(19) United States
(12) Patent Application Publication
(10) Pub. No.: US 2008/0312215 A1
(43) Pub. Date: Dec. 18, 2008

(54) Substituted [1,4]-diazepanes as CXCR3 antagonists and their use in the treatment of inflammatory disorders

(75) Inventors: Andrew G. Cole, Robbinsville, NJ (US); Marc-Raleigh Brescia, Dayton, NJ (US); Ian Henderson, Hopewell, NJ (US)

Correspondence Address: HESLIN ROTHENBERG FARLEY & MESITI PC, 5 COLUMBIA CIRCLE, ALBANY, NY, 12203, US

(73) Assignee: Pharmacopeia Drug Discovery, Inc., Princeton, NJ (US)

(21) Appl. No.: 11/427,175

(22) Filed: Jun. 28, 2006

Related U.S. Application Data
(60) Provisional application No. 60/694,477, filed on Jun. 28, 2005.

(51) Int. Cl. A61K 31/551; C07D 243/08; A61P 19/02; A61P 1/00

(52) U.S. Cl. 514/218; 540/575

(57) ABSTRACT

CXCR3 inhibitors of formula

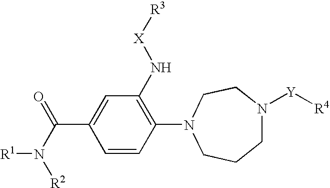

are disclosed. Inhibition of CXCR3 activation is useful for treating disorders resulting from CXCR3-associated T-cell mediated function, such as inflammatory bowel disease, multiple sclerosis, rheumatoid arthritis and diabetes, as well as in the prevention of allograft rejection. N-ethyl-1,4-diazepane-1-carboxamides in which R¹ is substituted or unsubstituted arylalkyl and R³ is substituted or unsubstituted aryl are particularly preferred.

### CROSS-REFERENCE TO RELATED APPLICATIONS

This application claims priority to U.S. provisional application Ser. No. 60/694,477 filed Jun. 28, 2005, the entire contents of which are hereby incorporated by reference.

### FIELD OF THE INVENTION

The invention relates to substituted [1,4]-diazepanes that are CXCR3, receptor antagonists. The compounds, and pharmaceutically acceptable salts thereof, are useful for the treatment of disorders that are mediated by CXCR3 function.

### BACKGROUND OF THE INVENTION

Chemokines are cytokines that play an important role inflammatory and immune response. Chemokines are divided into four major groups (CXC, CC, C and CX3C) based on the structural separation of conserved cysteine residues within the peptide sequence. CXC and, CX3C (all of which display four conserved cysteine residues) display one and three amino acid residues, respectively, between the first and second conserved cysteine residues whereas the CC chemokines display sequential cysteine residues. C chemokines exhibit only two conserved cysteine residues (the second and fourth cysteine residues, within other groups) (Murphy et al, Pharmacol. Rev. 2000, 52, 145).

Chemokine receptors are members of the super family of G-protein coupled receptors (GPCR's) having seven transmembrane-spanning regions. The natural chemokine ligands for CXCR3, Mig (monokine induced by interferon-γ/CXCL9), IP-10 (interferon-inducible protein 10/CXCL10) and I-TAC (interferon-inducible T cell a chemoattractant/CXCL11), are thought to play a key role in directing activated T cells and other cell types (such as NK cells) to sites of inflammation.

The CXCR3 receptor has been implicated in Th1 cell-mediated inflammation; CXCR3 is one of the most abundant chemokine receptors on Th1 cells (reviewed in Annunziato et al, Eur Cytokine Netw. 1998, 9, 12). Consequently, inhibition of chemokine function via CXCR3 may be useful for the treatment of a number of disorders relating to T cell-mediated function, including inflammatory bowel disease, multiple sclerosis, rheumatoid arthritis, and diabetes, as well as in the prevention of allograft rejection. CXCR3-bearing T-lymphocytes are enriched in inflamed intestinal tissue (Papadakis K. et al, Inflammatory bowel diseases 2004, 10, 778; Yuan et al, Inflammatory bowel diseases 2001, 7, 281) and CXCR3 ligands IP-10 and Mig are expressed in inflamed tissues in mucosal immune, responses (Singh et al, Journal of Interferon and Cytokine Research 2003, 23, 591). Antibodies against IP-10 have been shown to inhibit inflammation in two mouse models of colitis (Sasaki et al, European Journal of Immunology 2002, 32, 3197; Singh et al, Journal of Immunology 2003, 171, 140). Blockade of IP-10 was also effective against disease symptoms and T-cell proliferation in two animal models of multiple sclerosis (mouse hepatitis virus infection and experimental allergic encephalomyelitis (EAE); reviewed in Tsunoda et al., Mult. Scler. 2004, 10, 26 and Arimilli et al, Immunol. Rev. 2000, 177, 43). CXCR3 plays a role in insulin-dependent diabetes (reviewed in Arimilli et al, Immunol Rev. 2000, 177, 43) and CXCR3 ligands secreted by pancreatic beta cells are chemoattractants for infiltrating T-cells in insulitis. (Frigerio et al, Nat. Med. 2002, 8, 1414). Both CXCR3 (Motoki et al, Modern Rheumatology, 2003, 13, 114; Lande et al, Journal of Immunology 2004, 173, 2815; Qin et al, Journal of Clinical Investigation 1998, 101, 746) and its ligands (Patel et al, Clinical Immunology 2001, 98, 39) are upregulated in synovial fluid and/or peripheral blood in rheumatoid arthritis. Allograft survival is prolonged in acute graft rejection models in CXCR3- or IP-10-deficient mice or in the presence of antibodies directed against the receptor or IP-10 (Hancock et al, J. Exp. Med. 2000, 192, 1515; Hancock et al, J. Exp. Med. 2001, 193, 975; Baker et al, Surgery 2003, 134, 126; the potential uses of CXCR3 antagonists for prevention of graft rejection are reviewed in Vincenti et al, Am. J. Transplant 2002, 2, 898).

In addition to its role in inflammation, CXCR3 has been implicated in angiogenesis and its role has been reported to be either angiogenic or angiostatic. Postischemic neovascularization is decreased in CXCR3-deficient mice (Waeckel et al, Circulation-Research 2005, 96, 576). However, the receptor has more often been observed to have an angiostatic effect (Luster et al, J. Exp. Med. 1995, 182, 219; Strieter et al, J. Biol. Chem., 1995, 270, 27348; Arenberg et al, J. Leukoc. Biol. 1997, 62, 554; reviewed in Rosenkilde and Schwartz, APMIS 2004, 112, 481) and expression of the receptor in endothelial cells is cell cycle-regulated (Romagnani et al, J. Clin. Invest. 2001, 107, 53).

### SUMMARY OF THE INVENTION

In one aspect, the invention relates to a genus of CXCR3 inhibitors sharing the general formula I:

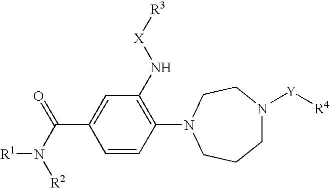

wherein:
R¹ is substituted or unsubstituted alkyl, substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;

### R² is H;

### X is CO—, or (CO)—NH—;

R³ is substituted or unsubstituted C2-C6 alkyl, substituted or unsubstituted cycloalkyl, substituted or unsubstituted aryl, substituted or unsubstituted heteroaryl, substituted or unsubstituted heterocycle substituted or unsubstituted arylalkyl substituted or unsubstituted heteroarylalkyl;

### Y is H, C(O)—, CON—, or C(O)NH—; and

R⁴ is H, or substituted or unsubstituted alkyl,
wherein R³ is not pyridine when R¹ is alkyl.

In another aspect, the invention relates to a method of treating a condition associated with CXCR3 function comprising administering to a subject in need of such treatment a therapeutically effective amount of a compound of formula I

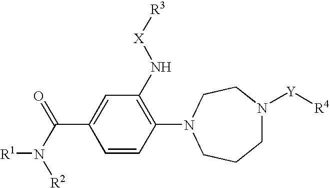

wherein:
R¹ is substituted or unsubstituted alkyl substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;

### R² is H;

### X is CO—, or (CO)—NH—;

R³ is substituted or unsubstituted C2-C6 alkyl, substituted or unsubstituted cycloalkyl, substituted or unsubstituted aryl, substituted or unsubstituted heteroaryl, substituted or unsubstituted-heterocycle, substituted or unsubstituted arylalkyl, substituted or unsubstituted heteroarylalkyl;

### Y is H, C(O)—, CON—, or C(O)NH—; and

R⁴ is H, or substituted or unsubstituted alkyl,
wherein R³ is not pyridine when R¹ is alkyl
or a pharmaceutically acceptable salt thereof.

Such conditions include inflammatory bowel disease, multiple sclerosis, rheumatoid arthritis, diabetes and allograft rejection.

In another aspect, the invention relates to pharmaceutical compositions comprising a pharmaceutically acceptable carrier and compounds of formula I, including pharmaceutically acceptable salts thereof, in any stereoisomeric form, or a mixture of any such compounds in any ratio. The compositions may comprise an additional anti-inflammatory agent.

### DETAILED DESCRIPTION OF THE INVENTION

In the description that follows, certain conventions will be followed as regards the usage of terminology including the abbreviations and definitions described below unless otherwise stated:
Ac—Acetyl
BSA—Bovine Serum Albumin
Boc—tert-butoxycarbonyl
Boc₂O—tert-butoxycarbonic anhydride
C—carbon
c—cyclo
δ—Nuclear Magnetic Resonance chemical shift referenced to tetramethylsilane
DCE—1,2-dichloroethane
DCM—dichloromethane=methylene chloride=CH₂Cl₂
DIPEA—Diisopropylethylamine
DMAP—4-Dimethylamino pyridine
DMF—N,N-Dimethylformamide
DMSO—Dimethyl sulfoxide
EDC—1-(3-dimethylaminopropyl)-3-ethylcarbodiimide hydrochloride
Et—Ethyl
EtOAc—Ethyl acetate
Et₃N—Triethylamine
FLIPR—Fluorometric Imaging Plate Reader, Molecular Devices
¹H NMR—Proton Nuclear Magnetic Resonance
HATU—O-(7-Azobenzotriazol-1-yl)-1,1,3,3-tetramethyluronium hexafluorophosphate
HBSS—Hanks Balanced Salt Solution
HEPES—4-(2-hydroxyethyl)-1-piperazineethanesulfonic acid
Hexanes—HPLC grade isomeric hexanes
HOBt—Hydroxybenzotriazole
i—so
IP-10—interferon-inducible protein 10/CXCL10
I-TAC—interferon-inducible T cell a chemoattractant/CXCL11
LCMS—Liquid Chromatography Mass Spectroscopy
m—-meta
Me—Methyl
MeOH—Methanol
Mig—monokine induced by interferon-γ/CXCL9
min—minutes
n—normal
N—Nitrogen
NMR—Nuclear Magnetic Resonance
NaCNBH₃—Sodium cyano borohydride
Na(OAc)₃BH—Sodium triacetoxy borohydride
o—-ortho
p—-para
Ph—Phenyl
r.t.—room temperature
sat.—saturated
s—secondary
t—tertiary
TFA—Trifluoro acetic acid
THF—Tetrahydrofuran

### DEFINITIONS

“Alkyl” refers to C1-C10 substituted, branched, unsubstituted and linear hydrocarbons potentially substituted at any of the C1-C10 positions. Examples of alkyl groups include but are not limited to methyl, ethyl, n-propyl, i-propyl, n-butyl, t-butyl- and t-butyl, pentyl, hexyl, octyl and the like.

“Cycloalkyl” refers to C3-C10 substituted or unsubstituted cyclic hydrocarbons potentially substituted at any of the C3-C10 positions. “Cycloalkyl” includes groups involving cyclic hydrocarbon functionality as a substitution of an alkyl group. Examples of cycloalkyl groups include but are not limited to c-propyl, c-butyl, c-pentyl, c-hexyl, and the like.

“Alkoxy” refers to alkoxy groups from 1 to 8 carbon atoms of a straight, branched, cyclic configuration and combinations thereof. Examples of alkoxy groups include, but are not limited to methoxy, ethoxy, propoxy, isopropoxy, cyclopropyloxy, and the like.

Halogen includes F, Cl, Br, and I, with F and Cl as the preferred groups.

“Aryl” refers to C6-C14 substituted or unsubstituted unsaturated aromatic carbocycle containing single or multiple rings. Examples of aryl groups include, but are not limited to phenyl, napthyl, biphenyl and the like.

“Arylalkyl” refers to an alkyl containing an aryl ring. Examples of arylalkyl groups include, but are not limited to benzyl, phenethyl, phenylpropyl, phenylbutyl and the like. Arylalkyl groups can be substituted or unsubstituted. Substitution can be incorporated at positions within the aryl segment of arylalkyl, the alkyl segment of arylalkyl, and combinations thereof.

“Heteroaryl” refers to C3-C10 aryl ring(s) containing one or more heteroatoms selected from nitrogen, oxygen and sulfur, within the ring(s) in a heteroaromatic system. Heteroaryl can be monocyclic or poly cyclic, with monocyclic and bicyclic preferred. Rings can be substituted or unsubstituted. Examples of ring substituents include but are not limited to alkyl, substituted alkyl, cycloalkyl, alkoxy, aryl, heteroaryl, heterocycle, carbonyl, carboxy, NO₂, halogen, hydroxy, cyano, benzyl, phenoxy, naphthyloxy, aryloxy, benzyloxy and the like.

“Heterocycle” refers to a C3-C10 aromatic or non aromatic ring systems comprising monocyclic or poly cyclic ring systems containing one or more heteroatoms selected from nitrogen, oxygen and sulfur, within the ring(s). Rings can be substituted or unsubstituted.

“Heteroarylalkyl” refers to an alkyl containing a heteroaryl ring. Examples of heteroarylalkyl groups include, but are not limited to furfuryl, thiophene methyl, thiophene ethyl, pyridine methyl, pyridine ethyl and the like. The term oxygen or sulfur heteroarylalkyl refers to groups in which the heteroaryl ring contains an oxygen or sulfur but not nitrogen, for example, furanylalkyl and thiophenealkyl. Heteroarylalkyl can be present as different isomers, for example, but not limiting, 2-, 3- and 4-pyridine methyl heteroarylalkyl groups can be substituted or unsubstituted. Substitution can be incorporated at positions with in the aryl segment of heteroarylalkyl, the alkyl, segment of heteroarylalkyl, and combinations thereof.

Arylcycloalkyl refers to an aryl group fused to a cycloalkyl group, the two having two atoms in common. Substitution can be incorporated at positions within the aryl segment of arylcycloalkyl, the alkyl segment of arylcycloalkyl, and combinations thereof.

Groups that are termed to be “substituted” may be substituted in any manner with single or multiple substituents in such a way that the substitution does not adversely affect the desired activity of compounds of type I. Examples of substitution are detailed in the detailed description of the invention and examples, and may include but are not limited to alkyl, cycloalkyl, alkoxy, alkylaryl, aryl, heteroaryl, alkylheteroaryl, heterocycle, carbonyl, sulfonyl, carboxy, carboxyamido, amino (primary, secondary and tertiary, alkylamino, dialkylamino, arylamino, diarylamino, arylalkylamino, diarylalkylamino, heteroarylamino, diheteroarylamino, heteroarylalkylamino, diheteroarylalkylamino, alcohol, acyl, aroyl, heteroaroyl, nitro, cyano, keto, halogen, haloalkyl (for example trifluoromethyl), haloalkoxy (for example trifluoromethoxy), amino acyl, amino aroyl.

Some of the compounds described herein may contain one of more asymmetric centers and may thus give rise to enantiomers, diastereomers, and other stereoisometric forms which may be defined in terms of absolute stereochemistry as (R) or (S). The present invention is meant to include all such possible enantiomers and diastereomers and mixtures thereof. Optically active (R) and (S) isomers may be prepared using chiral synthons or chiral reagents, or resolved using conventional techniques

“Pharmaceutically acceptable salt” as used herein, refers to a composition involving a salt prepared from a pharmaceutically acceptable non-toxic organic or inorganic acid or base, including hydrates thereof. Pharmaceutically acceptable salts are known in the art.

The present invention provides substituted diazepanes as CXCR3 antagonists. Preferred compounds of the invention are found in the class of substituted diazepane carboxamides of the formula

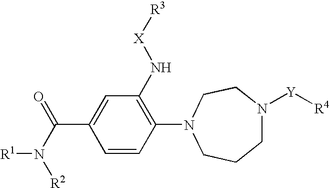

in which Y is C(O)NH, X is CO— and R² is H. Exemplary compounds are shown in Table 1. Details with respect to synthesis and analysis of the compounds of the invention are provided below.

### Analysis

Analysis of the compounds of the invention was performed by analytical HPLC according to one of two methods:

Method A employed a Waters Millenium 2690/996PDA separations system employing a Phenomonex Luna 3u C8(2) 50×4.6 mm analytical column. The aqueous acetonitrile based solvent gradient involves;

0-1 min—Isocratic 5% of (0.05% TFA/acetonitrile);

1 min-7 min—Linear gradient of 5-90% of (0.05% TFA/acetonitrile);

7 min-9 min—Isocratic 90% of (0.05% TFA/acetonitrile);

9 min-10 min—Linear gradient of 90-5% of (0.05% TFA/acetonitrile);

10 min-12 min—Isocratic 5% of (0.05% TFA/acetonitrile).

Flow rate=1 mL/min.

Method B entailed analysis by a Millenium 2690/996PDA separations system employing a Phenomenex Columbus 5u c18 column 50×4.60 mm analytical column. The aqueous acetonitrile based solvent gradient involves;

0-0.5 min—Isocratic 10% of (0.05% TFA/acetonitrile);

0.5 min-5.5 min—Linear gradient of 10-90% of (0.05% TFA/acetonitrile):

5.5 min-7.5 min—Isocratic 90% of (0.05% TFA/acetonitrile);

7.5 min-8 min—Linear gradient of 90-10% of (0.05% TFA/acetonitrile);

8 min-10 min—Isocratic 10% of (0.05% TFA/acetonitrile).

Flow rate=0.4 mL/min.

Mass Spectroscopy was conducted using Thermo-electron LCQ classic.

Liquid Chromatography Mass Spectroscopy was conducted using a Waters Millenium 2690/996PDA linked Thermo-electron LCQ classic.

¹H NMR spectroscopy was conducted using a Varian 300 MHz Gemini 2000 FTNMR.

TABLE 1

| | | Mass Spec. | HPLC |
|---|---|---|---|
| Example | Structure | Found | (Minutes/method) |
| 1 | 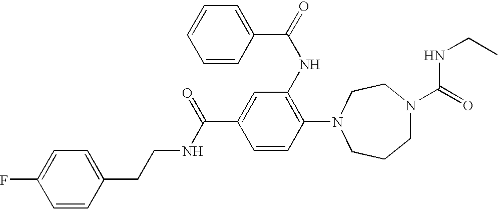 | 532 [M + H] | 6.46 min/A |
| 2 | 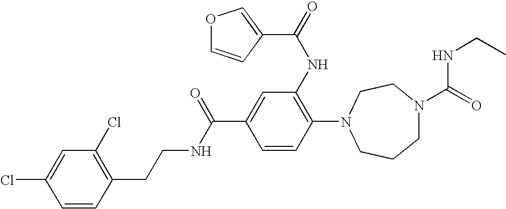 | 572 [M + H] | 6.79 min/A |
| 3 | 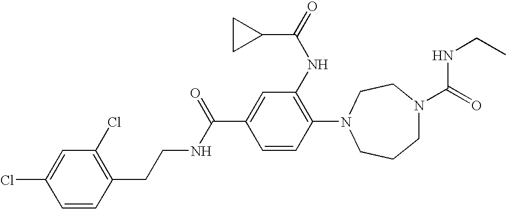 | 546 [M + H] | 6.67 min/A |
| 4 | 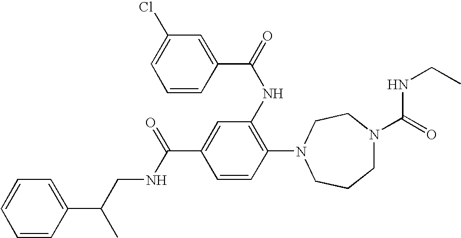 | 562 [M + H] | 7.03 min/A |
| 5 | 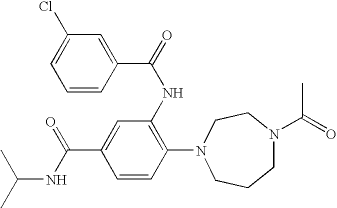 | 457 [M + H] | 5.95 min/A |
| 6 | 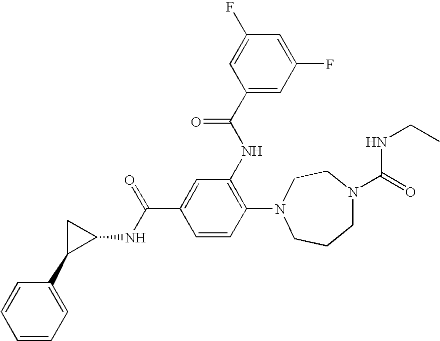 | 562 [M + H] | 6.87 min/A |
| 7 | 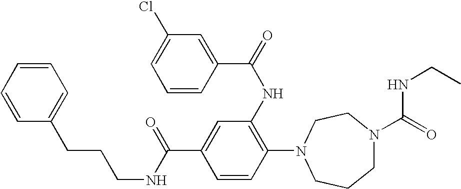 | 562 [M + H] | 7.07 min/A |
| 8 | 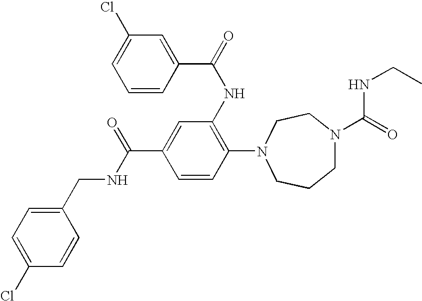 | 568 [M + H] | 7.05 min/A |
| 9 | 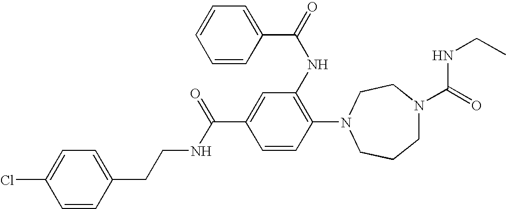 | 548 [M + H] | 6.75 min/A |
| 10 | 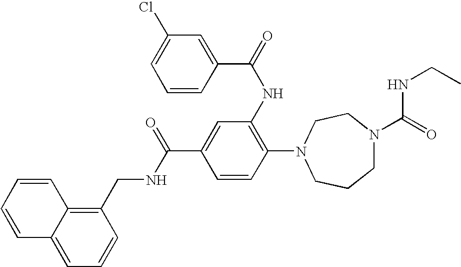 | 584 [M + H] | 7.21 min/A |
| 11 | 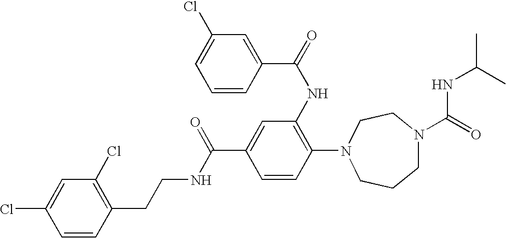 | 630 [M + H] | 7.81 min/A |
| 12 | 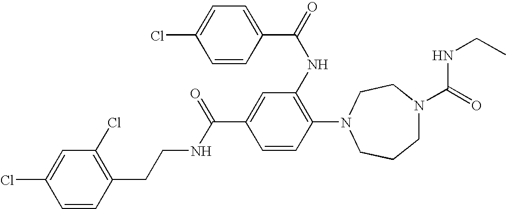 | 616 [M + H] | 7.53 min/A |
| 13 | 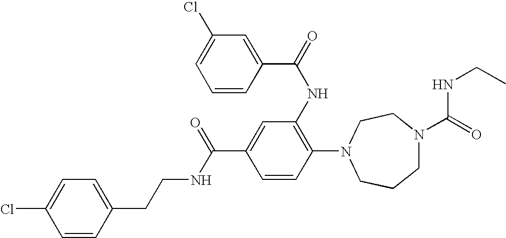 | 582 [M + H] | 7.24 min/A |
| 14 | 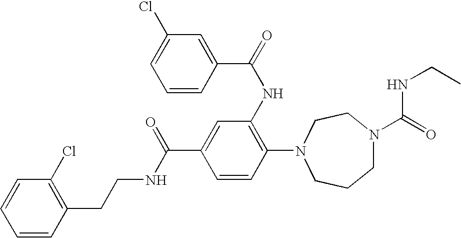 | 582 [M + H] | 7.18 min/A |
| 15 | 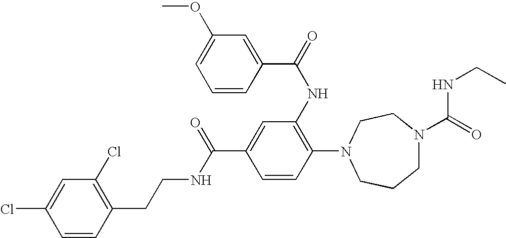 | 612 [M + H] | 7.23 min/A |
| 16 | 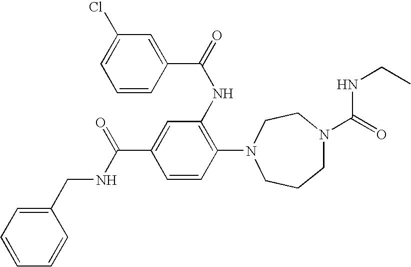 | 534 [M + H] | 6.76 min/A |
| 17 | 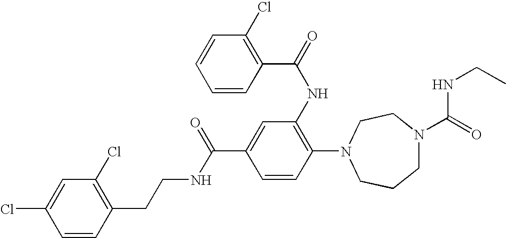 | 616 [M + H] | 7.28 min/A |
| 18 | 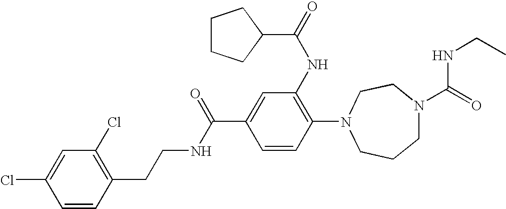 | 574 [M + H] | 7.10 min/A |
| 19 | 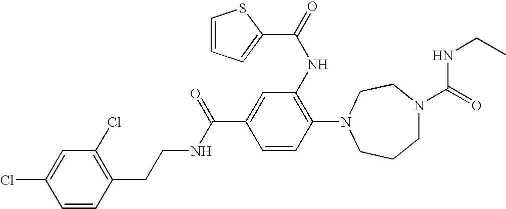 | 588 [M + H] | 7.12 min/A |
| 20 | 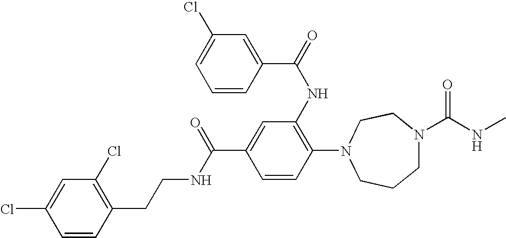 | 602 [M + H] | 7.27 min/A |
| 21 | 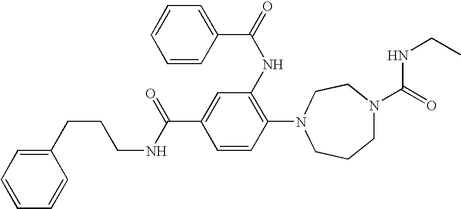 | 528 [M + H] | 6.68 min/A |
| 22 | 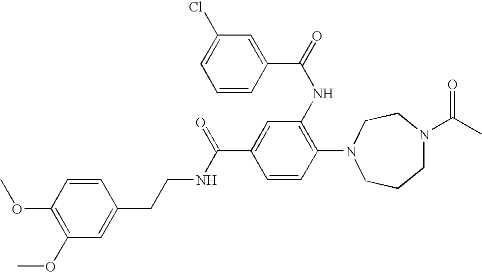 | 579 [M + H] | 6.19 min/A |
| 23 | 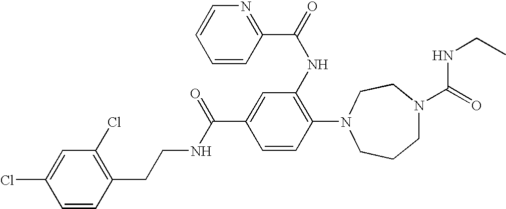 | 583 [M + H] | 7.16 min/A |
| 24 | 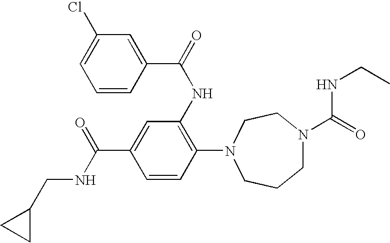 | 498 [M + H] | 6.25 min/A |
| 25 | 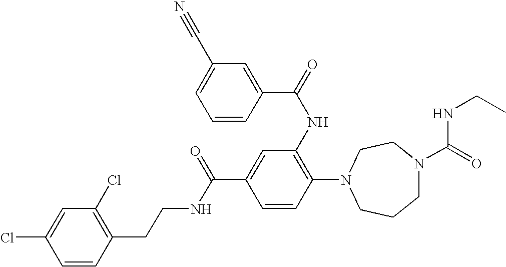 | 607 [M + H] | 7.02 min/A |
| 26 | 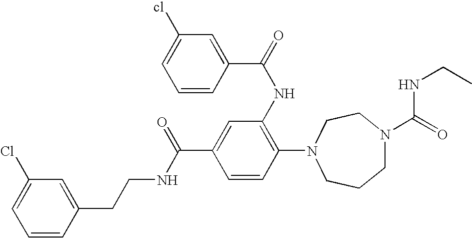 | 582 [M + H] | 7.13 min/A |
| 27 | 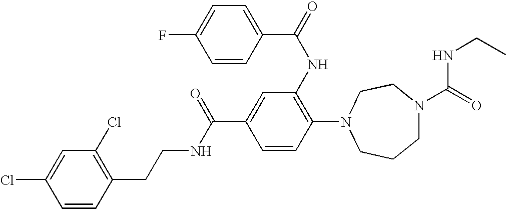 | 600 [M + H] | 7.29 min/A |
| 28 | 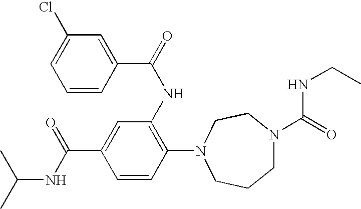 | 486 [M + H] | 6.23 min/A |
| 29 | 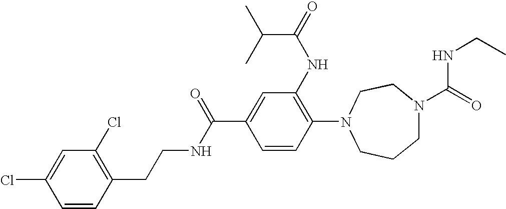 | 548 [M + H] | 6.80 min/A |
| 30 | 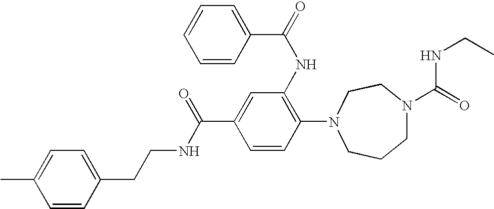 | 528 [M + H] | 6.65 min/A |
| 31 | 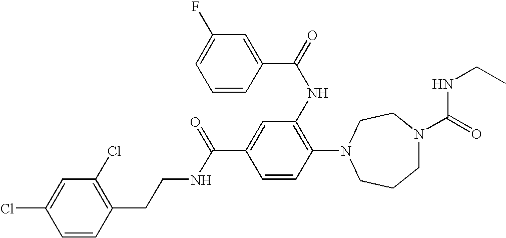 | 600 [M + H] | 7.30 min/A |
| 32 | 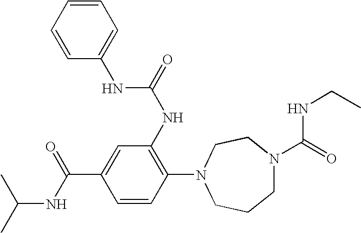 | 467 [M + H] | 6.53 min/A |
| 33 | 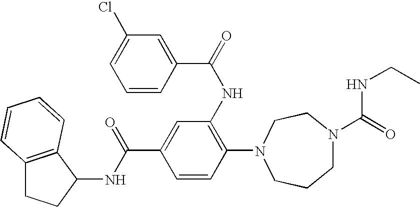 | 560 [M + H] | 7.00 min/A |
| 34 | 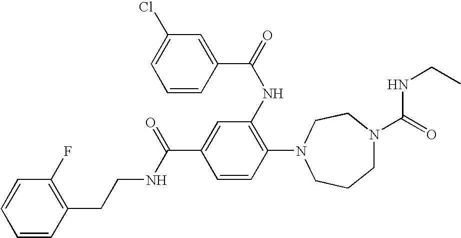 | 566 [M + H] | 6.94 min/A |
| 35 | 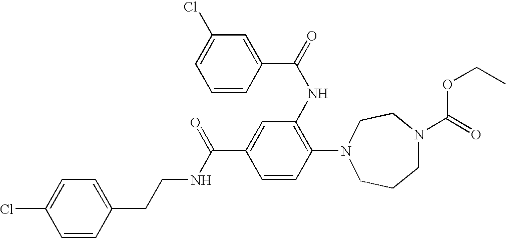 | 583 [M + H] | 7.76 min/A |
| 36 | 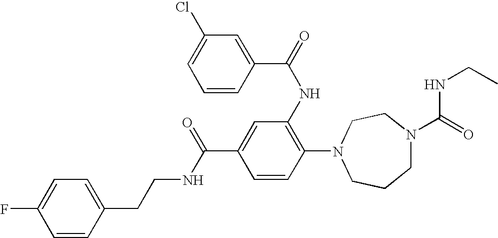 | 566 [M + H] | 6.93 min/A |
| 37 | 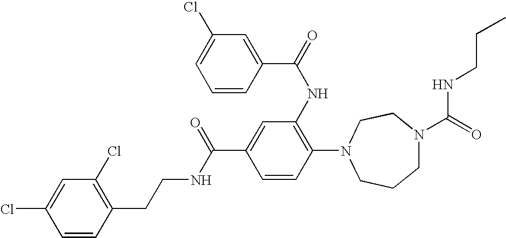 | 630 [M + H] | 7.77 min/A |
| 38 | 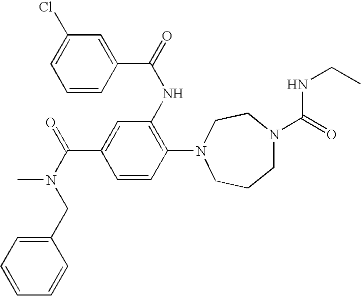 | 548 [M + H] | 6.94 min/A |
| 39 | 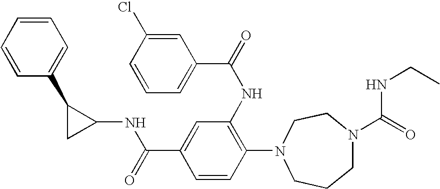 | 560 [M + H] | 7.03 min/A |
| 40 | 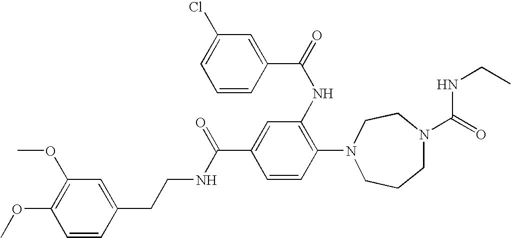 | 608 [M + H] | 6.54 min/A |
| 41 | 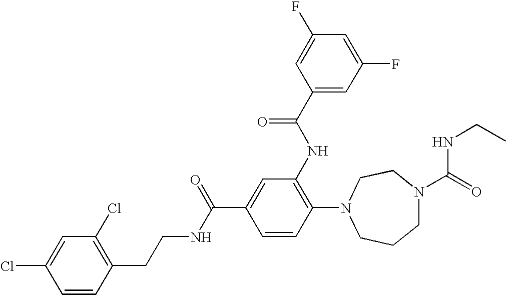 | 618 [M + H] | 7.40 min/A |
| 42 | 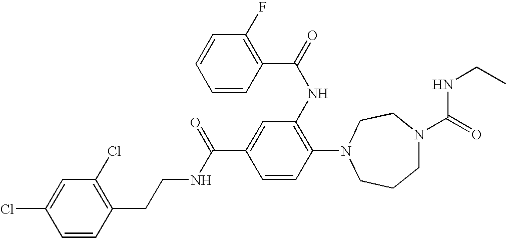 | 600 [M + H] | 7.42 min/A |
| 43 | 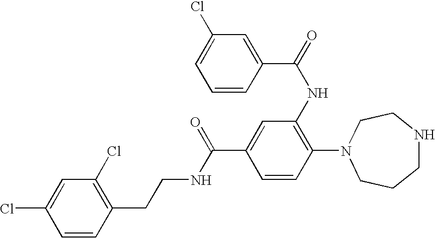 | 545 [M + H] | 6.13 min/A |
| 44 | 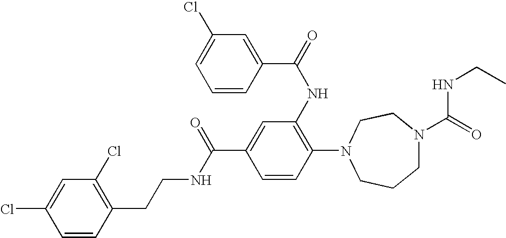 | 616 [M + H] | 7.56 min/A |
| 45 | 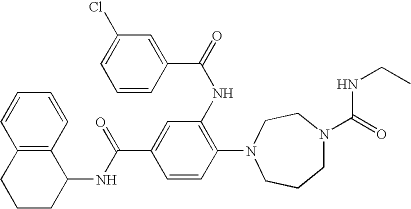 | 574 [M + H] | 7.20 min/A |
| 46 | 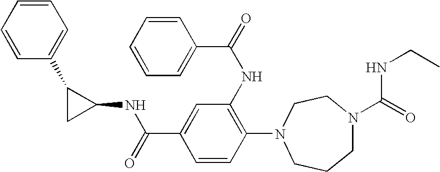 | 526 [M + H] | 6.59 min/A |
| 47 | 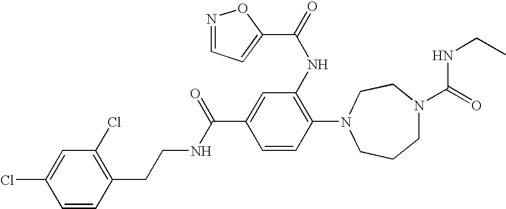 | 573 [M + H] | 6.73 min/A |
| 48 | 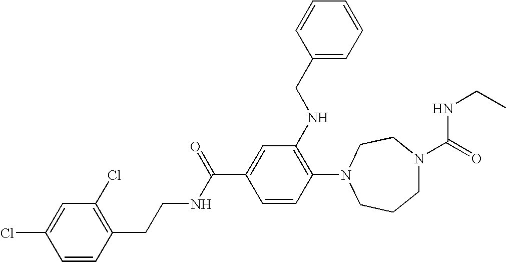 | 568 [M + H] | 7.44 min/A |
| 49 | 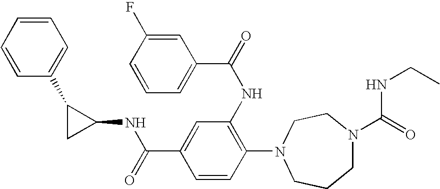 | 544 [M + H] | 7.74 min/A |
| 50 | 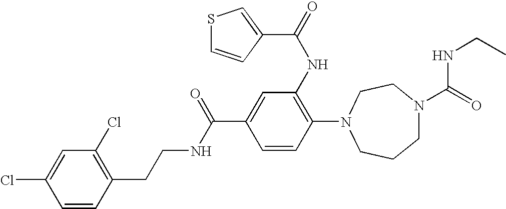 | 588 [M + H] | 6.92 min/A |
| 51 | 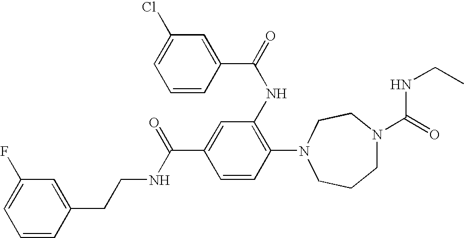 | 566 [M + H] | 6.97 min/A |
| 52 | 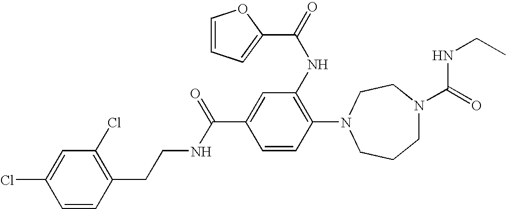 | 572 [M + H] | 6.89 min/A |
| 53 | 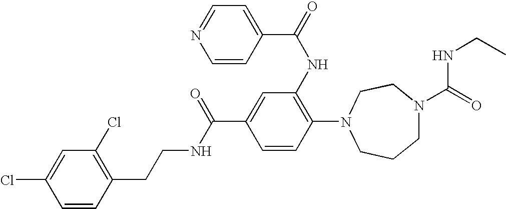 | 583 [M + H] | 5.61 min/A |
| 54 | 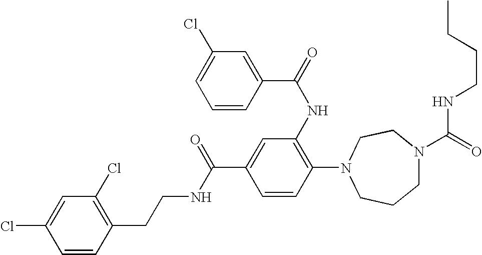 | 644 [M + H] | 8.12 min/A |
| 55 | 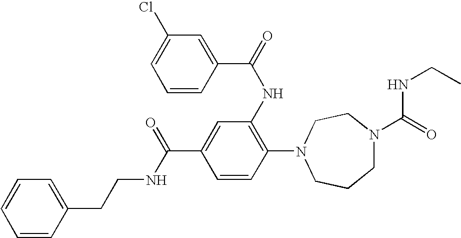 | 548 [M + H] | 6.90 min/A |
| 56 | 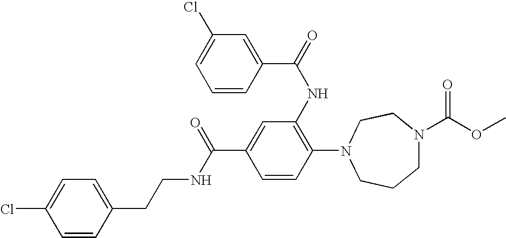 | 569 [M + H] | 7.51 min/B |
| 57 | 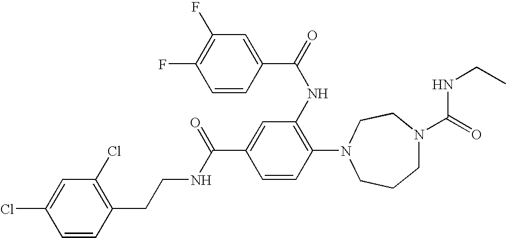 | 618 [M + H] | 7.43 min/A |
| 58 | 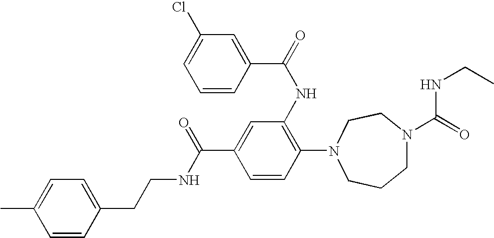 | 562 [M + H] | 7.11 min/A |
| 59 | 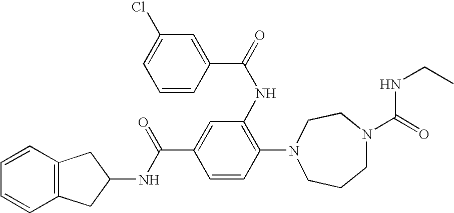 | 560 [M + H] | 6.95 min/A |
| 60 | 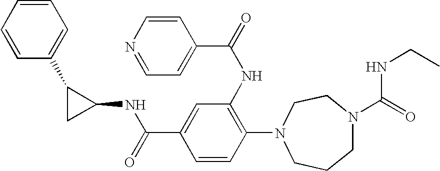 | 527 [M + H] | 5.18 min/A |
| 61 | 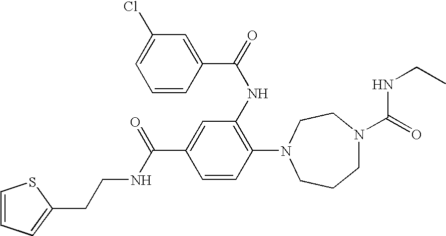 | 554 [M + H] | 6.28 min/A |
| 62 | 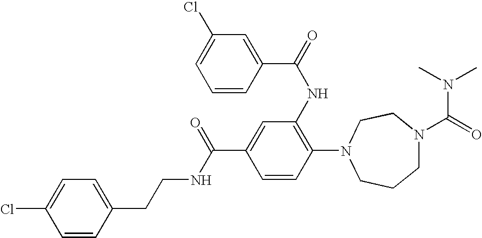 | 582 [M + H] | 7.29 min/B |
| 63 | 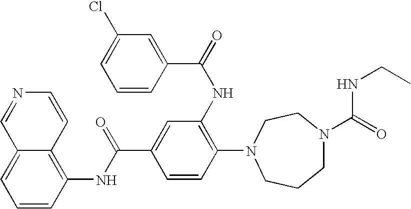 | 571 [M + H] | 5.08 min/A |
| 64 | 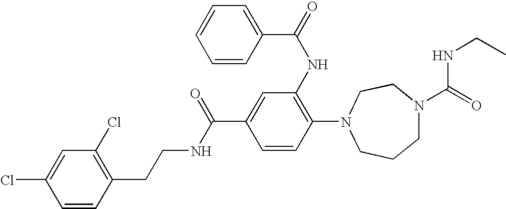 | 582 [M + H] | 7.16 min/A |

### EXPERIMENTAL

Compounds of type I can be synthesized by means of conventional organic synthesis employing solid-phase and solution phase chemistries. By way of illustration, but not limitation, the synthesis of compounds of type I is detailed in schemes 1 and 2.

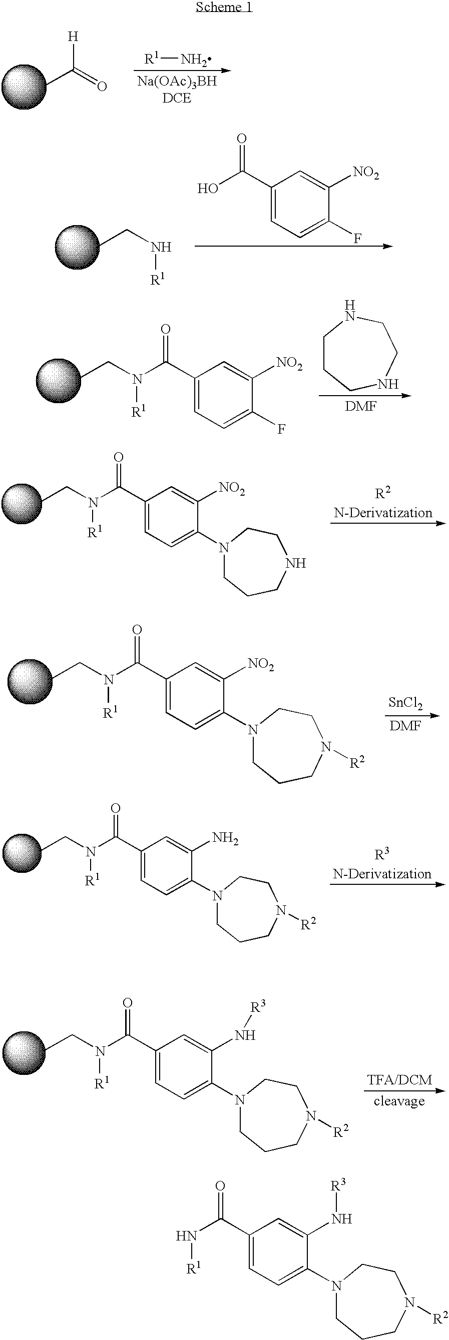

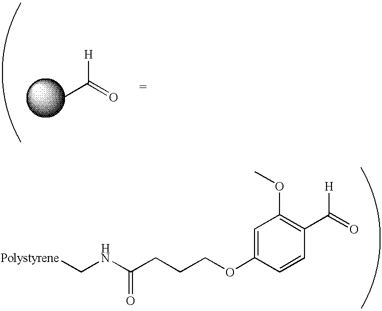

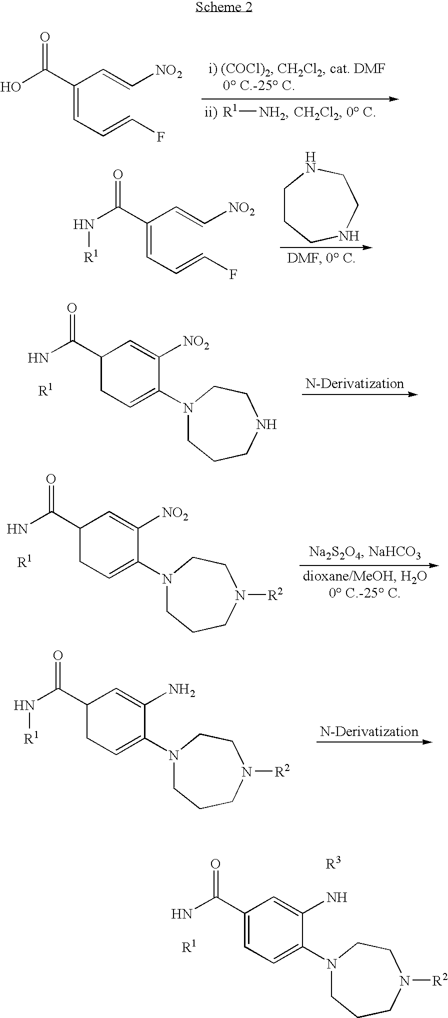

### Solid-Phase Synthesis of Compounds of Type I

Compounds of type I can be synthesized on solid-phase in five steps from 4-(4′-formyl-3′-methoxy)phenoxybutyric acid functionalized amino methyl terminated polystyrene resin utilizing commercially available 4-nitro-3-fluoro benzoic acid (Scheme 1). Reductive alkylation onto the formyl group of the acid labile linker, followed by amide formation with 4-nitro-3-fluoro benzoic acid provides the carboxamide. Fluoro displacement with an excess of homopiperazine to provide the N-aryl[1,4]-diazepane is followed by urea formation with an isocyanate or an N-carbamoyl chloride, carbamate formation with a chloroformate, amide formation with an anhydride or an acid chloride. Tin chloride mediated nitro-reduction and subsequent N-derivatization of the resulting primary aniline with an acid chloride to provide the amide or reductive alkylation to provide the amino derivative or urea formation with an isocyanate provides compounds of type I. Ligand cleavage from the solid support is achieved using TFA in CH₂Cl₂, allowing compound purification by flash chromatography or preperative HPLC.

### Solid-Phase Synthesis—General Procedures

For solid-phase reactions it is often desirable to think of the amount of solution reagents in terms of concentrations rather than equivalents. For this reason, reagent concentration is generally provided in the following experimental protocols. All shaking is performed with a wrist-action shaker. The size of shaking vessels typically employed is 20 mL (small) or 100 mL (medium). Each washing cycle is carried out with 12 mL of solvent for small shaking vessels or 60 mL of solvent for medium vessels over 5-10 minutes unless otherwise stated. All solvents used for reactions and washings are HPLC grade unless otherwise stated. Reactions which require heating are performed in scintillation vials with Teflon-lined screw caps. These are placed in an oil bath. Upon reaction completion, the resin in the scintillation vial is transferred to a glass shaking vessel and washed. The resin-bound ligand can be removed by acid cleavage with TFA/CH₂Cl₂.

### Intermediate 1 (I-1)—General Procedure A—Acylation with 4-(4′-formyl-3′-methoxy)phenoxybutyric acid

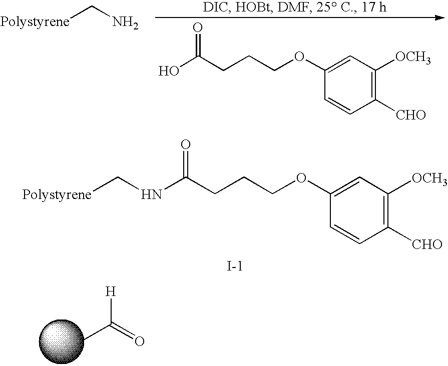

To a solution of 2.86 μg (12.0 mmol, 0.2 M, 4.0 eq.) of 4-(4′-formyl-3′-methoxy)phenoxybutyric acid and 1.84 g (12.0 mmol, 0.2 M, 4.0 eq.) of HOBt.H₂O in 60 mL of DMF was added 3.75 mL (24.0 mmol, 0.4 mL, 8.0 eq.) of DIC. The resulting solution was stirred for 20 min at 25° C. This solution was added to a medium shaking vessel containing 3.8 g (˜0.8 mmol/g, 3.0 mmol, 1.0 eq.) aminomethyl terminated Polystyrene. The mixture was shaken for 17 h at 25° C. The shaking vessel was then drained and the resin was washed with DMF (1×), CH₂Cl₂ (1×), DMF (2×), CH₂Cl₂ (2×), CH₃OH (2×) and CH₂Cl₂ (2×).

### Intermediate 2 (I-2)—General Procedure B—Reductive Amination

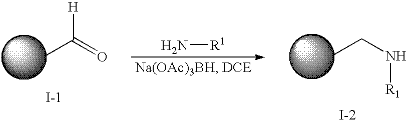

To a suspension of 0.6 g (40.8 mmol, 0.48 mmol, 1.0 eq.) of resin-bound o-methoxybenzaldehyde (I-1) in 12 mL of 1,2-dichloroethane (DCE) was added 4.8 mmol (0.4 M, 10.0 eq.) of a primary amine. The resin suspension was shaken for 15 sec and 1.0 g (4.8 mmol, 0.4 M, 10.0 eq.) of sodium triacetoxyborohydride was added. The suspension was shaken for 16 h at 25° C., venting the reaction vessel periodically during the first 1 h. The vessel was then drained, and the resin was washed with CH₃OH (1×), CH₂Cl₂ (2×), CH₃OH (1×), CH₂Cl₂ (2×), Cl₃OH (1×), CH₃OH (1×30 min) and CH₂Cl₂ (2×).

### Intermediate 3 (I-3)—General Procedure C—N-Acylation with 3-nitro-4-fluoro benzoic-acid

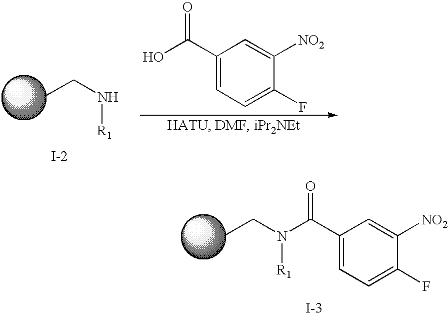

To 0.6 g (˜0.7 mmol/g, 0.4 mmol, 1.0 eq.) of resin-bound secondary amine (I-2) in 10 mL of DMF was added 0.46 g (2.5 mmol, 0.25 M, ˜3.5 eq.) of 3-nitro-4-fluoro benzoic acid and 0.95 g (2.5 mmol 0.25 M, ˜3.5 eq.) of HATU. A portion of 0.87 mL (5.0 mmol, 0.5 M, ˜7 eq.) of, N,N-diisopropylethylamine was added and the mixture was shaken at 25° C. for 16 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 4-(I-4)—General Procedure D—N-Arylation with homopiperazine

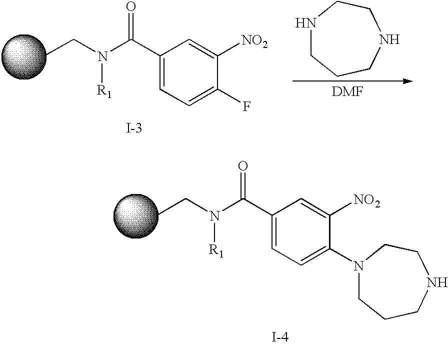

To 0.6 g (˜0.7 mmol/g, 0.4 mmol, 1.0 eq.) of resin-bound aryl fluoride (I-3) in 10 mL of DMF was added 0.5 g (5 mmol, 0.5 M, ˜7 eq.) of homopiperazine and the mixture was shaken at 25° C. for 16 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 5 (I-5)—General Procedure E—N-Derivatization—Urea Formation

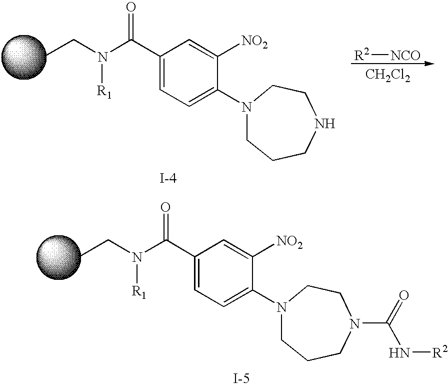

To 0.6 g (˜0.7 mmol/g, 0.4 mmol, 1.0 eq.) of resin-bound secondary amine (I-4) in 10 mL of CH₂Cl₂ was added 2.5 mmol (0.25 M, ˜3.5 eq.) of an isocyanate and the mixture was shaken at 25° C. for 16 h. The vessel was drained and the resin was washed with CH₂Cl₂ (1×), DMF (1×), CH₂Cl₂ (2×), CH₃OH (2×) and CH₂Cl₂ (2×).

### Intermediate 6 (I-6)—General Procedure F—Nitro Reduction

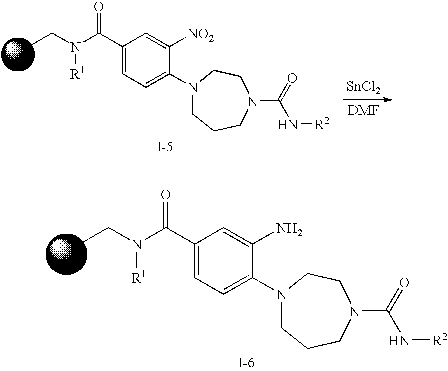

To 0.6 g (˜0.7 mmol/g, 0.4 mmol, 1.0 eq.) of resin-bound nitro compound (I-5) was added 10 mL of a 2 M solution of tin (II) chloride dihydrate in DMF and the mixture was shaken at 25° C. for 36 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 7 (I-7)—General Procedure G—N-Derivatization—Amide Formation

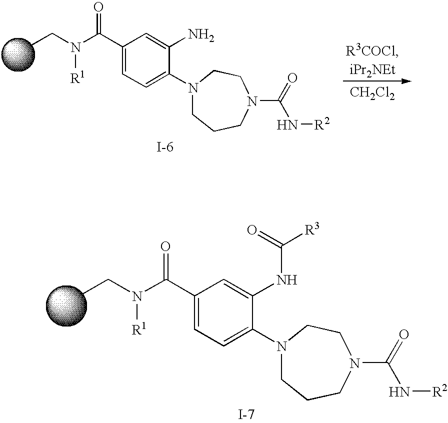

To 0.6 g (˜0.7 mmol/g, 0.4 mmol, 1.0 eq.) of resin-bound aniline (I-6) in 10 mL of CH₂Cl₂ was added 0.87 mL (5.0 mmol, 0.5 M, ˜7 eq.) of N,N-diisopropylethylamine and 2.5 mmol (0.25 M, ˜3.5 eq.) of an acid chloride. The mixture was shaken at 25° C. for 16 h. The vessel was drained and the resin was washed with CH₂Cl₂ (1×), DMF (1×), CH₂Cl₂ (2×), CH₃OH (2×) and CH₂Cl₂ (2×).

### Intermediate 8 (I-8)—General Procedure H—Acid Cleavase

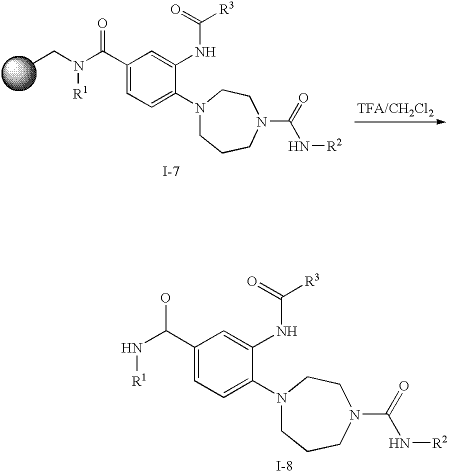

To 0.2 g of resin bound diazepane (I-7) in a scintillation vial was added 10 mL of 50% v/v TFA/CH₂Cl₂, and the resulting resin suspension was stirred at rt for 2 h. The resin was removed by filtration and the solvent removed in vacuo. The residue was purified by preparative HPLC.

### Solution-Phase Synthesis

Compounds of type I can be synthesized in five steps from commercially available 4-nitro-3-fluoro benzoic acid (Scheme 2). Activation of the carboxyl group as the acid chloride is followed by amide formation with an amine to provide the carboxamide. Fluoro displacement with an excess of homopiperazine to provide the N-aryl[1,4]-diazepane is followed by urea formation with an isocyanate or an N-carbamoyl chloride, carbamate formation with a chloroformate, amide formation with an anhydride or acid chloride. Nitro-reduction and subsequent N-derivatization of the resulting primary aniline with an acid chloride to provide the amide or reductive alkylation to provide the amino derivative or urea formation with an isocyanate to provide the urea results in the formation of compounds of type I. Analogous compounds of type I can be synthesised using similar experimental procedures.

### Intermediate 9 (I-9)—Procedure I: N-|2-(2,4-Dichloro-phenyl)-ethyl|-4-fluoro-3-nitro-benzamide

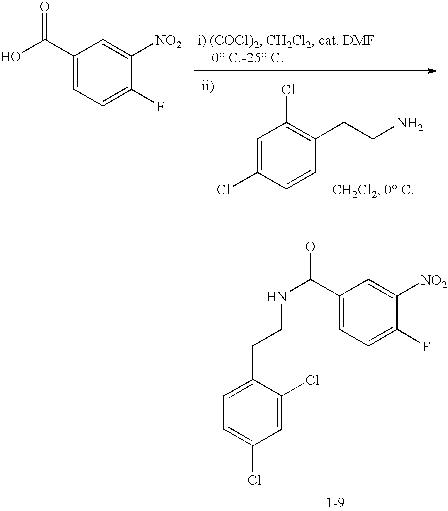

To a solution of 5.0 g (27.0 mmol, 1.0 eq) of 3-nitro-4-fluoro benzoic acid in 100 mL of CH₂Cl₂ at 0° C. was added 4.7 mL (54.0 mmol, 2.0 eq.) of oxalyl chloride and 100 μL (1.3 mmol, 0.05 eq.) of DMF. The resulting solution was stirred at 0° C. for 1 h, allowed to warm to room temperature and stirred for an additional 16 h. The solvent was removed in vacuo to provide 3-nitro-4-fluoro benzoyl chloride. The crude acid chloride was dissolved in 150 mL of CH₂Cl₂ and cooled to 0° C. A portion of 8.1 mL (54.0 mmol, 2.0 eq.) of 2-(2,4-dichlorophenyl)ethyl amine was added over 10 min, and the mixture stirred at 0° C. for 20 min. The reaction mixture was diluted with 300 mL of CH₂Cl₂, washed with 100 mL of 1M HCl, 80 mL of sat. NaHCO₃, dried (Na₂SO₄) and the solvent removed in vacuo to provide 9.5 g (26.6 mmol, 98%) of 1-9 as a yellow solid. (δ_{H}, 300 MHz, CDCl₃) 3.02 (t, 2H), 3.70 (q, 2H), 6.25 (bt, 1H), 7.10-7.40 (m, 4H), 8.04 (m, 1H), 8.39 (dd. 1H); ESI, 562 └M+H┘.

### Intermediate 10 (I-10)—Procedure J: 4-|1,4|Diazepan-1-yl-N-|2-(2,4-dichloro-phenyl)-ethyl|-3-nitro-benzamide

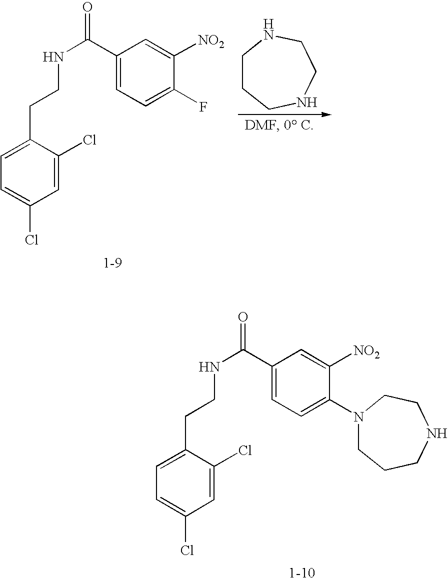

To a solution of 9.5 g (26.6 mmol. 1.0 eq.) of I-9 in 100 mL of DMF at 0° C. was added a solution of 8.1 g (79.8 mmol 3.0 eq.) of homopiperazine in 50 mL of DMF. The resulting red solution was stirred at 0° C. for 15 min and 150 mL of water added. The mixture was extracted with 3×100 mL of diethyl ether and the combined organic extracts were washed with 100 mL of sat. brine, dried (Na₂SO₄) and the solvent removed in vacuo to provide crude I-10, (δ_{II}, 300 MHz, CDCl₃) 1.95 (m, 2H), 2.91 (m, 2H), 3.03 (m, 4H), 3.29 (m, 2H), 3.46 (m, 2H), 3.66 (m, 2H), 6.22 (bt, 1H), 7.04 (d, 1H), 7.16 (m, 2H), 7.77 (dd, 1H), 8.06 (d, 1H).

### Intermediate 11 (I-11)—Procedure K: 4-{4-└2-(2,4-Dichloro-phenyl)-ethylcarbamoyl|-2-nitro-phenyl}-|1,4|diazepane-1-ethly urea

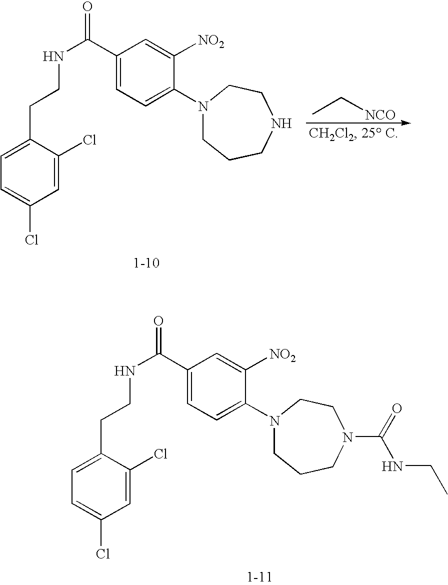

To a solution of 11.6 g (26.2 mmol, 1.0 eq.) of I-10 in 150 mL of CH₂Cl₂ was added 2.1 mL (29.3 mmol, 5.0 eq.) of ethyl isocyanate. The resulting solution was stirred at 25° C. for 30 min. The solvent was removed in vacuo to provide 10.5 g (20.6 mmol, 78% from I-9) of I-11 as a yellow solid. (δ_{II}, 300 MHz, CDCl₃) 1.05 (t, 3H), 1.95 (m, 2H), 3.02 (t, 2H), 3.18 (dq, 2H), 3.32 (m, 2H), 3.42 (m, 4H), 3.65 (m, 4H) 4.37 (bt, 1H), 6.43 (bt, 1H), 7.02 (d, 1H), 7.16 (m, 3H), 7.36 (m, 1H), 7.77 (dd, 1H), 8.05 (d, 1H)

### Intermediate 12 (I-12)—Procedure L: 4-{2-Amino-4-└2-(2,4-dichloro-phenyl)-ethylcarbamoyl|-phenyl}-|1,4|diazepane-1-ethly urea

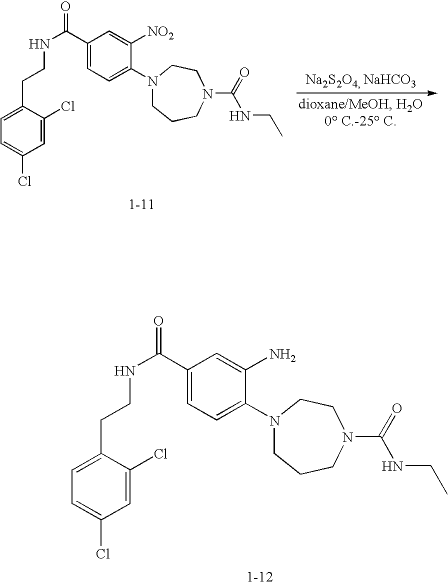

A solution of 7.8 g of sodium hydrosulfite (tech grade, ˜38 mmol, 3 eq.) and 2.5 g of sodium bicarbonate (29.7 mmol, 2.2 eq.) in 100 mL of water was added to a solution of 7.0 g of I-11 in 150 mL of 2:1 v/v p-dioxane/methanol at 0° C. over 10 min. The resulting suspension was allowed to warm to room temperature and stirred for an additional 30 min. The mixture was diluted with 250 mL of water and extracted with 3×150 mL of EtOAc. The combined organic extracts were dried (Na₂SO₄), the solvent removed in vacuo and the residue purified by flash column chromatograph (EtOAc to 10% MeOH/EtOAc) to provide 4.5 g (9.4 mmol, 68%) of I-12 as a white solid. (δ_{II}, 300 MHz, CDCl₃) 1.08 (t, 3H), 1.98 (m, 2H), 3.03 (m, 6H), 3.26 (dq, 2H), 3.53 (t, 2H), 3.62 (m, 4H), 4.02 (bs, 2H), 4.35 (t, 1H) 6.14 (bt, 1H), 6.92 (m, 2H), 7.15 (m, 3H), 7.40 (s, 1H).

### Intermediate 13 (I-13)—General Procedure M: N-Derivatization

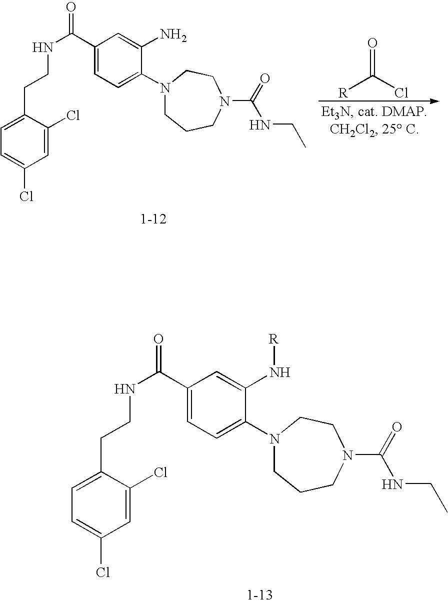

To a solution of 100 mg (0.21 mmol, 1.0 eq.) of I-12 in 2 mL of CH₂Cl₂ was added 72 μL of triethylamine (0.52 mmol, 2.5 eq.) and catalytic DMAP. A portion of 0.25 mmol (1.2 eq.) of an acid chloride was added and the resulting solution stirred at 25° C. for 1.5 h. The mixture was diluted 30 mL of CH₂Cl₂, washed with 10 mL of sat. NaHCO₃, dried (Na₂SO₄) and the solvent removed in vacuo. The residue was purified by flash column chromatography (EtOAc to 10% MeOH/EtOAc) to provide I-13.

### Intermediate 14 (I-14)—Procedure N: 4-Fluoro-3-nitro-benzoic acid methyl ester

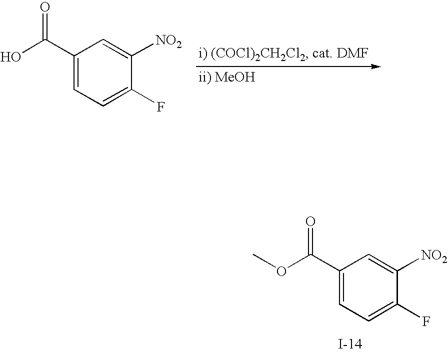

To a solution of 5.0 g (27.0 mmol, 1.0 eq) of 3-nitro-4-fluoro benzoic acid in 100 mL of CH₂Cl₂ at 0° C. was added 4.7 mL (54.0 mmol, 2.0 eq.) of oxalyl chloride and 100 μL (1.3 mmol, 0.05 eq.) of DMF. The resulting solution was stirred at 0° C. for 1 h, allowed to warm to room temperature and stirred for an additional 16 h. The solvent was removed in vacuo to provide 3-nitro-4-fluoro benzoyl chloride. The crude acid chloride was dissolved in 150 mL of MeOH at 0° C. and the mixture stirred for 20 min. The solvent was removed in vacuo to provide 9.5 g (26.6 mmol, 98%) of 4-Fluoro-3-nitro-methyl benzoate (I-14) as a white solid. (δ_{H}, 300 MHz, CDCl₃) 3.97 (s, 3H), 7.36 (dd, 1H), 8.30 (m, 1H), 8.73 (dd, 1H).

### Intermediate 15 (I-15)—Procedure O: 4-[1,4]Diazepan-1-yl-3-nitro-benzoic acid methyl ester

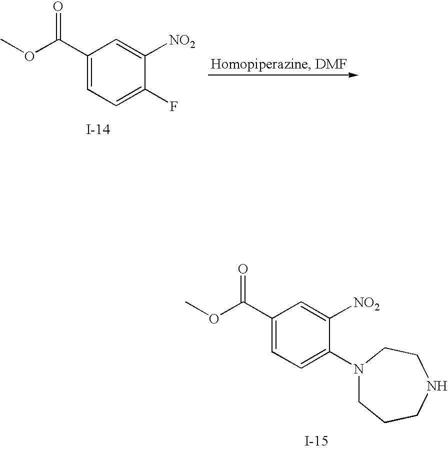

To a solution of 5.0 g (25.1 mmol, 1.0 eq.) of I-14 in 50 mL of DMF was added a solution of 12.6 g (125 mmol, 5.0 eq.) of homopiperazine in 100 mL of DMF. The resulting red solution was stirred at room temperature for 15 min and 150 mL of water added. The mixture was extracted with 3×100 mL of diethyl ether and the combined organic extracts were washed with 100 mL of sat. brine, dried (Na₂SO₄) and the solvent removed in vacuo to provide 6.5 g of crude I-15 as a yellow oil. (δ_{H}, 300 MHz, CDCl₃) 1.85 (m, 2H), 2.85 (m, 2H), 3.03 (m, 2H), 3.32 (m, 2H), 3.48 (m, 2H), 3.85 (s, 3H), 7.00 (d, 1H), 7.94 (dd, 1H), 8.36 (d, 1H), 8.39 (dd, 1H).

### Intermediate 16 (I-16)—Procedure P: 4-(4-Ethylcarbamoyl-[1,4]diazepan-1-yl)-3-nitro-benzoic acid methyl ester

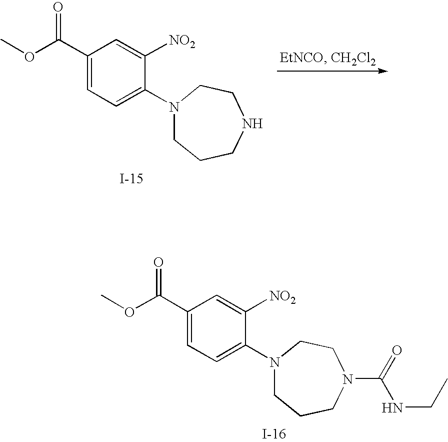

To a solution of 6.5 g (23.3 mmol, 1.0 eq.) of I-15 in 200 mL of CH₂Cl₂ was added 2.1 mL (29.3 mmol, 1.25 eq.) of ethyl isocyanate. The resulting solution was stirred at 25° C. for 30 min. The solvent was removed in vacuo to provide 8.5 g (24.3 mmol, 97% two steps) of I-16 as a deep yellow oil. (δ_{H}, 300 MHz, CD Cl₃) 1.04 (t, 3H), 1.95 (m, 2H), 3.20 (dq, 2H), 3.36 (m, 2H), 3.46 (m, 4H), 3.63 (m, 2H), 3.83 (s, 3H), 4.37 (t, 1H), 7.03 (d, 1H), 7.94 (d, 1H), 8.32 (d, 1H).

### Intermediate 17 (I-17)—Procedure Q: 3-Amino 4-(4-ethylcarbamoyl-[1,4]diazepan-1-yl)-benzoic acid methyl ester

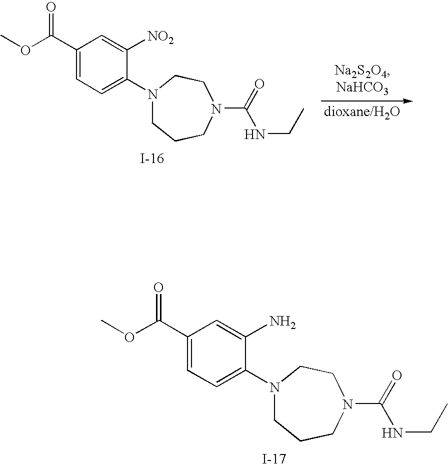

A solution of 17.7 g of sodium hydrosulfite (tech grade, ˜86 mmol, ˜5 eq.) and 5.7 g of sodium bicarbonate (67.6 mmol, 4.0 eq.) in 75 mL of water was added to a solution of 5.9 g (16.9 mmol, 1.0 eq.) of I-16 in 150 mL of p-dioxane at room temperature, over 15 min. The resulting suspension was stirred for an additional 30 min. The mixture was diluted with 200 mL of water and extracted with 3×150 mL of EtOAc. The combined organic extracts were dried (Na₂SO₄), the solvent removed in vacuo and the residue purified by flash column chromatography (EtOAc to 10% MeOH/EtOAc) to provide 4.2 g (13.1 mmol, 78%) of I-17 as a white solid. (δ_{H}, 300 MHz, CDCl₃) 1.12 (t, 3H), 1.98 (m, 2H), 3.10 (m, 4H), 3.30 (dq, 2H), 3.56 (t, 2H), 3.65 (m, 2H), 3.82 (s, 3H), 4.02 (s, 2H), 4.35 (t, 1H), 6.98 (d, 1H), 7.36 (s, 1H), 7.37 (dd, 1H).

### Intermediate 18 (I-18)—Procedure R: 3-(3-Chloro-benzoylamino)-4-(4-ethylcarbamoyl-[1,4]diazepan-1-yl)-benzoic acid methyl ester

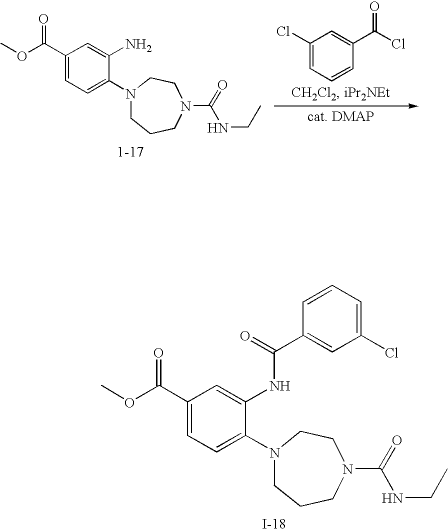

To a solution of 1.56 g (4.87 mmol, 1.0 eq.) of I-17 and 1.62 mL (11.67 mmol, 2.4 eq.) of triethylamine in 25 mL of CH₂Cl₂ was added 10 mg (cat.) of DMAP followed by 0.74 mL (5.85 mmol, 1.2 eq.) of 3-chlorobenzoyl chloride. The resulting mixture was stirred at room temperature for 1 hour and 50 mL of CH₂Cl₂ added. The organic solution was washed with 50 mL of water, 20 mL of sat. NaHCO₃, dried (Na₂SO₄), and the solvent removed in vacuo. The residue purified by flash column chromatography (80% EtOAc/hexanes to EtOAc) to provide 1.2 g (2.61 mmol, 54%) of I-18. (δ_{II}, 300 MHz, CDCl₃) 1.12 (t, 3H), 1.98 (m, 2H), 3.10 (m, 4H), 3.30 (dq, 2H), 3.56 (t, 2M, 3.65 (m, 2H), 3.82 (s, 3H), 4.02 (s, 2H), 4.35 (t 1H), 6.98 (d, 1H), 7.36 (s, 1H), 7.37 (dd, 1H). E.I. └M+H┘ 459.

### Intermediate 19 (I-19)—Procedure S: 3-(3-Chloro-benzoylamino)-4-(4-ethylcarbamoyl-|1,4|diazepan-1-yl)-benzoic acid

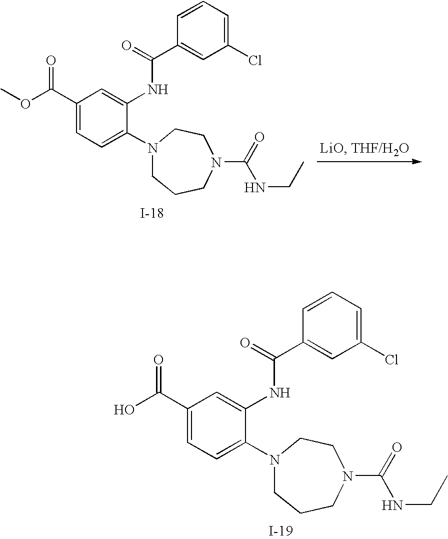

A solution of 0.21 g (8.7 mmol, 4.0 eq.) of lithium hydroxide in 10 mL of water was added to a solution of 1.0 g (2.18 mmol, 1.0 eq.) of I-18 in 10 mL of THF and the mixture stirred at 60° C. for 16 h. the mixture was cooled to room temperature and 50 mL of water added. The aqueous phase was acidified to pH 5 with 1M HCl. The product vas extracted into 3×50 mL EtOAc, the combined organic extracts dried (Na₂SO₄), removed in vacuo to provide 0.83 g (1.9 mmol, 86%) of I-19 as a white solid. (δ_{II}, 300 MHz. CDCl₃) 1.12 (t, 3H), 1.92 (m, 2H), 3.06 (m, 2H), 3.20 (m, 2H), 3.30 (q, 2H), 3.53 (t 2H), 3.68 (m, 2H). 7.15 (d, 1H), 7.48 (m, 2H), 7.77 (dd, 1H), 7.80 (d, 1H), 7.95 (t, 1H), 8.81 (d, 1H), 9.35 (bs, 1H). E.I. └M+H┘ 445.

### Intermediate 20 (I-20)—Procedure T: Amide Formation

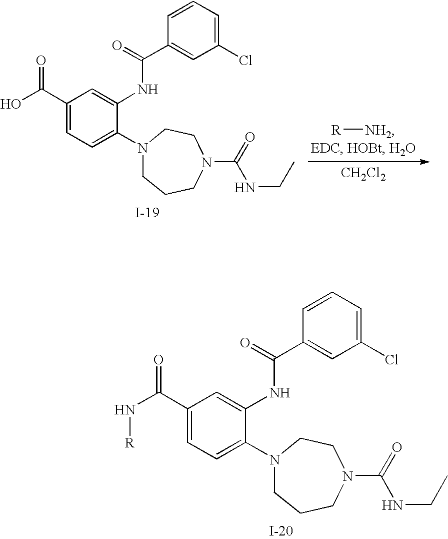

To a solution of 30 mg (0.07 mmol, 1.0 eq.) of I-19, 11 mg (0.08 mmol, 1.1 eq.) of HOBt and 15 mg (0.08 mmol, 1.1 eq.) of EDC in 2 mL or CH₂Cl₂ was, added 0.22 mmol (3.0 eq.) of an amine and the mixture stirred at room temperature for 2 h. The mixture was diluted with 20 mL of EtOAc, and washed with 10 mL of 1M HCl, 10 mL of sat. NaHCO₃, and 10 mL of sat. NaCl. The organic phase was dried (Na₂SO₄), and the solvent removed in vacuo. The residue purified by flash column chromatography or preperative HPLC to provide I-20.

### Representative Examples

### 4-(4-((4-fluorophenethyl)carbamoyl)-2-benzamidophenyl)-N-ethyl-1,4-diazepane-1-carboxamide

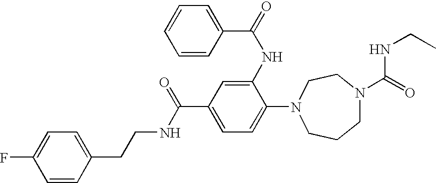

(δ_{II}, 300 MHz, CDCl₃) 1.21 (t, 3H), 2.12 (m, 2H), 3.02 (t, 2H), 3.16 (m, 2H), 3.22 (m, 2H), 3.39 (dq, 2H), 3.75 (m, 6H), 4.58 (bt, 1H) 6.68 (bt, 1H), 7.08 (t, 2H), 7.36 (m, 3H), 7.64 (m, 2H), 7.76 (dd, 1H), 8.00 (dd, 1H), 8.92 (d, 1H), 9.50 (bs, 1H); ESI, 532 └M+H┘.

### 4-(4-((2,4-dichlorophenethyl)carbamoyl)-2-(furan-4-carboxamido)phenyl)-N-ethyl-1,4-diazepane-1-carboxamide

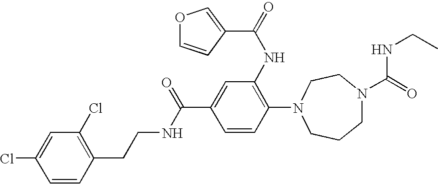

(δ_{H}, 300 MHz, CDCl₃) 1.22 (t, 3H), 2.12 (m, 2H), 3.09 (m, 4H), 3.28 (m, 2H), 3.42 (dq, 2H), 3.74 (m, 2H), 3.80 (m, 4H), 4.47 (bt, 1H) 6.50 (bt, 1H), 6.83 (d, 1H), 7.40 (m, 3H), 7.53 (s, 1H), 7.64 (m, 1H), 7.79 (dd, 1H), 8.21 (d, 1H), 8.83 (d, 1H), 9.04 (bs, 1H); ESI, 572 └M+H┘

### 4-(4-((2,4-dichlorophenethyl)carbamoyl)-2-(cyclopropanecarboxamido)phenyl)-N-ethyl-1,4-diazepane-1-carboxamide

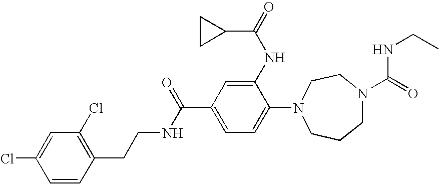

(δ_{H}, 300 MHz, CDCl₃) 1.00 (m, 2H), 1.11 (m, 2H), 1.24 (t, 3H), 2.01 (m, 1H), 2.13 (m, 2H), 3.12 (t, 2H), 3.21 (m, 4H), 3.42 (dq, 2H), 3.64 (t, 2H), 3.79 (m, 4H), 4.60 (bt, 1H) 6.56 (bt, 1H), 7.25 (m, 3H), 7.46 (d, 1H), 7.74 (dd, 1H), 8.72 (d, 1H), 8.84 (bs, 1H); ESI, 546 [M+H]

### (+/−)-4-(2-(3-chlorobenzamido)-4-((2-phenylpropyl)carbamoyl)phenyl)-N-ethyl-1,4-diazepane-1-carboxamide

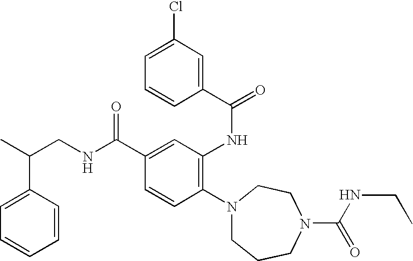

(δ_{H}, 300 MHz, CDCl₃) 1.25 (t, 3H), 1.48 (d, 3H), 2.12 (m, 2H), 3.15 (m, 5H), 3.40 (dq, 2H), 3.58 (m, 1H), 3.71 (t, 2H), 3.80 (m, 2H), 3.88 (m, 1H), 4.45 (t, 1H) 6.37 (bt, 1H), 7.40 (m, 5H), 7.65 (m, 4H), 7.84 (dt, 1H), 8.05 (m, 1H), 8.83 (d, 1H), 9.48 (bs, 1H); ESI, 562 [M+H].

### 4-(2-(3-chlorobenzamido)-4-(isopropylcarbamoyl)phenyl)-acetyl-1,4-diazepane

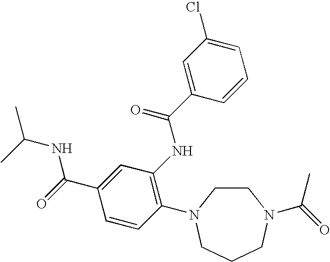

(δ_{H}, 300 MHz, CD₃OD) 1.38 (d, 6H), 2.05 (m, 2H), 2.10 and 2.22 (2s, 3H), 3.28 (m, 2H), 3.40 (m, 2H), 3.80 (m, 4H), 4.30 (m, 1H), 7.39 (m, 1H), 7.70 (m, 3H), 8.01 (m, 1H), 8.12 (m, 1H), 8.25 (dd, 1H); ESI, 457 [M+H].

### 4-(4-(((+/−)trans-2-phenylcyclopropyl)carbamoyl)-2-(3,5-difluorobenzamido)phenyl)-N-ethyl-1,4-diazepane-1-carboxamide

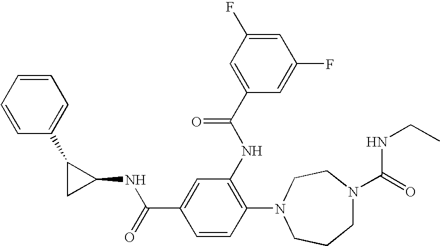

(δ_{H}, 300 MHz, CDCl₃) 1.27 (t, 3H), 1.45 (m, 3H), 2.15 (m, 2), 2.32 (m, 1H, 3.20 (m 3H), 3.26 (m, 2H), 3.40 (dq, 2H), 3.70 (t, 2H), 3.83 (m, 2H), 4.48 (t, 1H) 6.73 (bd, 1H), 7.15 (tt, 1H), 7.40 (m, 6H), 7.53 (m, 2H), 7.86 (dd, 1H), 8.85 (d, 1H), 9.48 (bs, 1H); ESI, 562 [M+H].

### 4-(2-(3-chlorobenzamido)-4-((3-phenylpropyl)carbamoyl)phenyl)-N-ethyl-1,4-diazepane-1-carboxamide

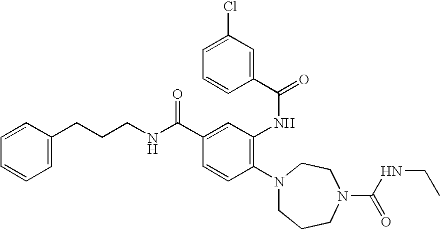

(δ_{H}, 300 MHz, CDCl₃) 1.26 (t 3H), 2.12 (m, 4H), 2.85 (t 2H), 3.20 (m, 2H), 3.28 (m, 2H), 3.41 (dq, 2H), 3.62 (q, 2H), 3.73 (t, 2H), 3.81 (m, 2H), 4.45 (t 1H), 6.40 (bt, 1H), 7.35 (m, 6H), 7.65 (m, 2H), 7.79 (dd, 1H), 7.87 (dt, 2H), 8.91 (d, 1H), 9.52 (bs, 1H); ESI, 562 [M+H].

### 4-(4-((4-chlorobenzyl)carbamoyl)-2-(3-chlorobenzamido)phenyl)-N-ethyl-1,4-diazepane-1-carboxamide

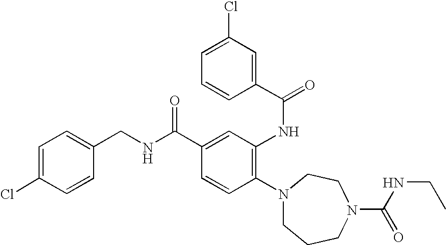

(δ_{H}, 300 MHz, CDCl₃) 1.21 (t 3H), 2.13 (m, 2H), 3.18 (m, 2H), 3.24 (m, 2H), 3.71 (dq, 2H), 3.71 (t, 2H), 3.80 (m, 2H), 4.52 (bt, 1H) 4.71 (d, 2H), 6.87 (bt, 1H), 7.38 (m, 5H), 7.61 (m, 2H); 7.82 (dd, 2H), 8.00 (dd, 1H), 8.97 (d, 1H), 9.50 (bs, 1H); ESL 568 [M+H].

### 4-(4-((4-chlorophenethyl)carbamoyl)-2-benzamidophenyl)-N-ethyl-1,4-diazepane-1-carboxamide

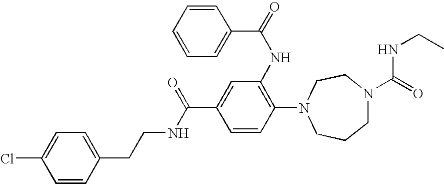

(δ_{H}, 300 MHz, CDCl₃) 1.21 (t, 3H), 2.08 (m, 2H), 3.02 (t, 2H), 3.15 (m, 2H), 3.25 (m, 2H), 3.38 (dq, 2H), 3.72 (m, 6H), 4.58 (bt, 1H), 4.71 (d, 2H), 6.72 (bt, 1H), 7.27 (m, 6H), 8.00 (dd, 1H), 8.92 (d, 1H)) 9.50 (bs, 1H); ESI, 548 [M+H].

### 4-(2-(3-chlorobenzamido)-4-((naphthalen-1-ylmethyl)carbamoyl)phenyl)-N-ethyl-1,4-diazepane-1-carboxamide

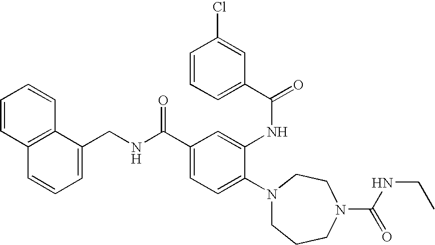

(δ_{H}, 300 MHz, CDCl₃) 1.23 (t, 3H), 2.12 (m, 2H), 3.17 (m, 2H), 3.23 (m, 2H), 3.39 (dq, 2H), 3.72 (t, 2H), 3.80 (m, 2H), 4.41 (bt, 1H), 5.22 (d, 2H), 6.63 (bt, 1H), 7.60 (m, 7H), 7.82 (dt, 2H), 7.97 (m, 3H), 8.22 (d, 1H), 9.47 (bs, 1H); ESI, 584 [M+H].

### 4-(4-((2,4-dichlorophenethyl)carbamoyl)-2-(3-chlorobenzamido)phenyl)-N-isopropyl-1,4-diazepane-1-carboxamide

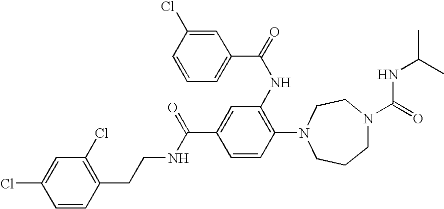

(δ_{H}, 300 MHz, CDCl₃) 1.21 (d, 6H), 2.12 (m, 2H), 3.17 (m, 4H), 3.24 (m, 2H), 3.71 (t, 2H), 3.80 (m, 1H), 4.31 (bd, 1H), 6.48 (bt, 1H), 7.35 (m, 3H), 7.51 (d, 1H), 7.62 (m, 2H), 7.78 (dd, 1H), 7.84 (d, 1H), 8.02 (d, 1H), 8.90 (d, 1H), 9.52 (bs, 1H); ESI, 630 [M+H].

### 4-(4-((2,4-dichlorophenethyl)carbamoyl)-2-(4-chlorobenzamido)phenyl)-N-ethyl-1,4-diazepane-1-carboxamide

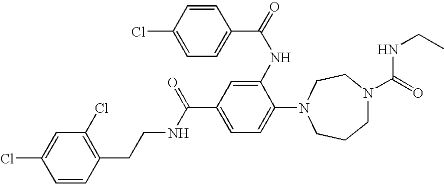

(δ_{II}, 300 MHz, CDCl₃) 1.21 (t, 3H), 2.04 (m, 2H), 3.12 (t, 4H), 3.23 (m, 2H), 3.38 (dq, 2H), 3.68 (m, 6H), 4.56 (bt, 1H), 6.60 (bt, 1H), 7.35 (m, 3H), 7.48 (s, 1H), 7.62 (d, 2H), 7.75 (dd, 1H), 7.95 (d, 2H), 8.89 (d, 1H), 9.49 (bs, 1H); ESI, 616 └M+H┘.

### 4-(4-((4-chlorophenethyl)carbamoyl)-2-(3-chlorobenzamido)phenyl)-N-ethyl-1,4-diazepane-1-carboxamide

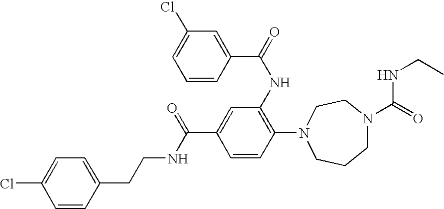

(δ_{H}, 300 MHz, CDCl₃) 1.23 (t, 3H), 2.12 (m, 2H), 3.71 (t, 2H), 3.18 (m, 2H), 3.24 (m, 2H), 3.40 (dq, 2H), 3.80 (m, 6H), 4.49 (bt, 1H), 6.52 (bt, 1H), 7.35 (m, 5H), 7.62 (m, 2H), 7.77 (dd, 2H), 7.83 (d, 1H), 8.02 (d, 1H), 8.87 (d, 1H), 9.53 (bs, 1H); ESI, 582 ℑM+H┘.

### 4-(4-((2-chlorophenethyl)carbamoyl)-2-(3-chlorobenzamido)phenyl)-N-ethyl-1,4-diazepane-1-carboxamide

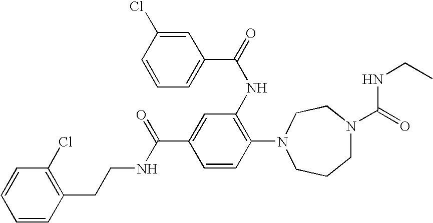

(δ_{H}, 300 MHz, CDCl₃) 1.23 (t, 3H), 2.12 (m, 1H), 3.18 (m, 6H), 3.40 (dq, 2H); 3.71 (t, 2H), 3.80 (m, 4H), 4.52 (bt, 1H), 6.59 (bt 1H), 7.35 (m 5H), 7.62 (m, 2H), 7.77 (dd, 1H), 7.83 (d, 1H), 8.02 (d, 1H), 8.87 (d, 1H), 9.53 (bs, 1H); ESI, 582 └M+H┘.

### 4-(4-((2,4-dichlorophenethyl)carbamoyl)-2-(3-methoxybenzamido)phenyl)-N-ethyl-1,4-diazepane-1-carboxamide

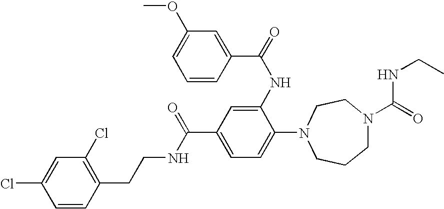

(δ_{H}, 300 MHz 9, CDCl₃) 1.22 (t 3H), 2.12 (m, 2H), 3.16 (m, 4H), 3.22 (m, 2H), 3.39 (dq, 2H), 3.72 (t, 2H), 3.80 (m, 4H), 4.00 (s, 3H), 4.52 (bt, 1H), 6.61 (bt, 1H), 7.21 (dd, 1H), 7.30 (m, 3H), 7.52 (m, 4H), 7.79 (dd, 1H), 8.92 (d, 1H), 8.87 (d, 1H), 9.50 (bs, 1H); ESI, 612 [M+H].

### 4-(4-(benzylcarbamoyl)-2-(3-chlorobenzamido)phenyl)-N-ethyl-1,4-diazepane-1-carboxamide

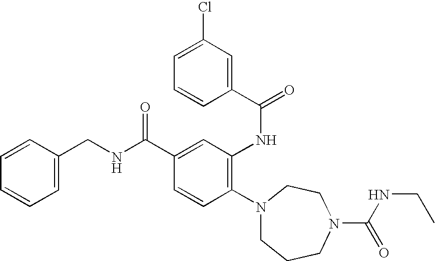

(δ_{H}, 300 MHz, CDCl₃) 1.22 (t, 3H), 2.15 (m, 2H), 3.18 (m, 2H), 3.25 (m, 2H), 3.39 (dq, 2H), 3.72 (t, 2H), 3.80 (m, 4H), 4.58 (bt, 1H), 4.78 (d, 2H), 6.79 (bt, 1H), 7.40 (m, 6H), 7.60 (m, 2H), 7.82 (m, 2H), 8.02 (d, 1H), 8.96 (d, 1H), 9.53 (bs, 1H), ESI 534 [M+H].

### 4-(4-((2,4-dichlorophenethyl)carbamoyl)-2-(2-chlorobenzamido)phenyl)-N-ethyl-1,4-diazepane-1-carboxamide

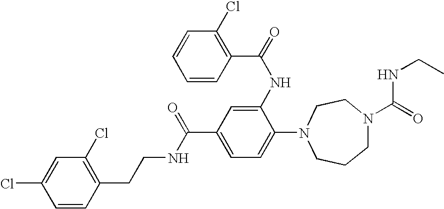

(δ_{H}, 300 MHz, CDCl₃) 1.22 (t, 3H), 2.05 (m, 2H), 3.16 (m, 6H), 3.37 (dq, 2H), 3.61 (t, 2H), 3.72 (m, 2H), 3.79 (q, 2H), 4.47 (bt, 1H), 6.61 (bt, 1H), 7.38 (m, 3H), 7.58 (m, 4H), 7.78 (dd, 1H), 7.88 (dd, 1H), 8.97 (d, 1H), 9.40 (bs, 1H); ESI, 616 [M+H].

### 4-(4-((2,4-dichlorophenethyl)carbamoyl)-2-(thiophene-2-carboxamido)phenyl)-N-ethyl-1,4-diazepane-1-carboxamide

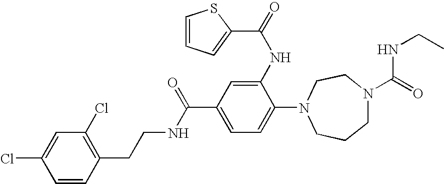

(δ_{H}, 300 MHz, CDCl₃) 1.23 (t 3H), 2.18 (m, 2H), 3.18 (m, 4H), 3.24 (m, 2H), 3.41 (dq, 2H), 3.80 (m, 6H), 4.50 (bt, 1H), 6.52 (bt, 1H), 7.38 (m, 4H), 7.50 (d, 1H), 7.69 (d, 1H), 7.80 (m, 2H), 8.84 (d, 1H), 9.39 (bs, 1H); ESI, 588 └M+H┘.

### 4-(4-((2,4-dichlorophenethyl)carbamoyl)-2-(3-chlorobenzamido)phenyl)-N-methyl-1,4-diazepane-1-carboxamide

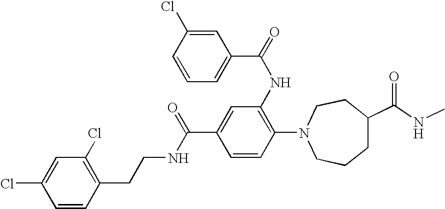

(δ_{H}, 300 MHz, CDCl₃) 2.13 (m, 2H), 2.91 (d, 3H), 3.19 (m, 4H), 3.24 (m, 2H), 3.71 (t, 2H), 3.80 (m, 4H), 4.58 (bq, 1H), 6.58 (bt, 1H), 7.38 (m, 2H), 7.50 (d, 1H), 7.64 (m, 2H), 7.75 (dd, 2H), 7.83 (dd, 1H), 8.02 (d, 1H), 8.88 (d, 1H); 9.50 (bs, 1H); ESI 602 [M+H].

### 4-(2-benzamido-4-((3-phenylpropyl)carbamoyl)phenyl)-N-ethyl-1,4-diazepane-1-carboxamide

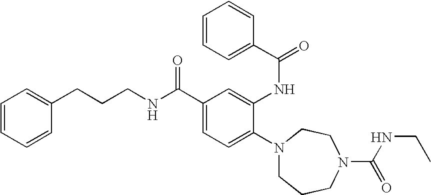

(δ_{H}, 300 MHz, CDCl₃) 1.22 (t, 3H), 2.08 (m, 4H), 2.81 (t, 2H), 3.15 (m, 2H), 3.23 (m, 2H), 3.39 (dq, 2H), 3.59 (q, 2H), 3.70 (t, 2H), 3.78 (m, 2H), 4.60 (bt, 1H), 6.62 (bt, 1H), 7.38 (m, 6H), 7.62 (m, 3H), 7.76 (dd, 1H), 8.00 (d, 2H), 8.92 (d, 1H), 9.53 (bs, 1H); ESI 528 [M+H].

### 4-(2-(3-chlorobenzamido)-4-(3,4-dimethoxyphenethylcarbamoyl)phenyl)acetyl-1,4-diazepane

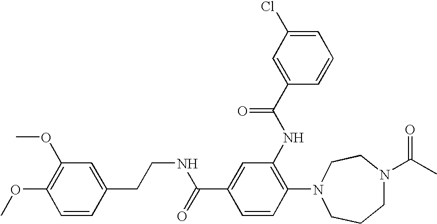

(δ_{H}, 300 MHz, CD₃OD) 2.03 (m, 2H), 2.08 and 2.22 (2s, 3H), 2.95 (t, 2H), 3.30 (m, 2H), 3.66 (t, 2H), 3.75 (t 2H), 3.82 (m, 4H) 3.86 (s, 3H), 3.89 (s, 3H), 6.95 (m, 3H), 7.38 (m, 1H), 7.70 (m, 3H), 8.00 (m, 1H), 8.12 (m, 1H), 8.30 (dd, 1H); ESI 579 [M+H].

### 4-(4-((2,4-dichlorophenethyl)carbamoyl)-2-(3-cyanobenzamido)phenyl)-N-ethyl-1,4-diazepane-1-carboxamide

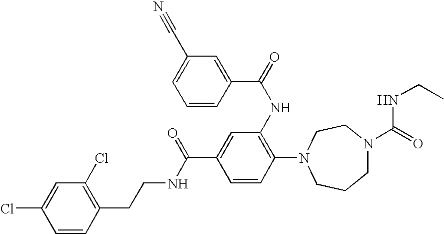

(δ_{H}, 300 MHz, CDCl₃) 1.10 (t, 3H), 2.97 (m, 2H), 3.06 (m, 4H), 3.15 (m, 2H), 3.25 (m, 2H), 3.57 (t, 2H), 3.67 (m, 4H), 4.36 (t, 1H), 6.32 (t 1H), 7.19 (m, 2H), 7.27 (m, 1H), 7.38 (d, 1H), 7.68 (m, 2H), 7.83 (m, 1H), 8.07 (m, 1H), 8.23 (m, 1H), 8.72 (d, 1H), 9.43 (bs, 1H); ESI, 607 [M+H].

### 4-(4-((3-chlorophenethyl)carbamoyl)-2-(3-chlorobenzamido)phenyl)-N-ethyl-1,4-diazepane-1-carboxamide

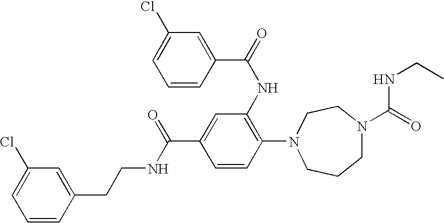

(δ_{II}, 300 MHz, CDCl₃) 1.25 (t, 3H), 2.14 (m, 2H), 3.05 (m, 2H), 3.19 (m, 2H), 3.28 (m, 2H), 3.40 (dq, 2H), 3.72 (t, 2H), 3.81 (m, 4H), 4.43 (t, 1H), 6.50 (t, 1H), 7.26 (m, 1H), 7.40 (m, 5H), 7.65 (m, 2H), 7.79 (dd, 1H), 7.86 (dt, 1H), 8.05 (m, 1H), 8.89 (d, 1H), 9.51 (bs, 1H); ESI, 582 └M+H┘.

### 4-(4-((4-chloro-2-methylphenethyl)carbamoyl)-2-(4-fluorobenzamido)phenyl)-N-ethyl-1,4-diazepane-1-carboxamide

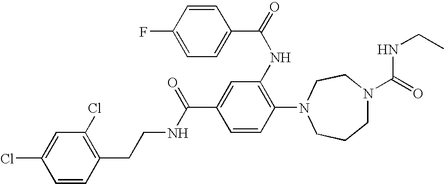

(δ_{II}, 300 MHz, CDCl₃) 1.22 (t, 3H), 2.10 (m, 1H), 3.12 (m, 4H), 3.22 (m, 2H), 3.38 (dq, 2H), 3.64 (t, 2H), 3.76 (m, 4H), 4.60 (t, 1H), 6.67 (t, 1H), 7.30 (m, 5H), 7.48 (d, 1H), 7.75 (dd, 1H), 8.00 (m, 2H), 8.86 (d, 1H), 9.47 (bs, 1H); ESI, 600 └M+H┘.

### 4-(2-(3-chlorobenzamido)-4-(isopropylcarbamoyl)phenyl)-N-ethyl-1,4-diazepane-1-carboxamide

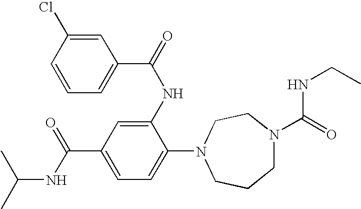

(δ_{II}, 300 MHz, CDCl₃) 1.24 (t, 3H), 1.40 (d, 6H), 2.10 (m, 2H), 3.16 (m, 2H), 3.24 (m, 2H), 3.40 (m, 2H), 3.72 (m, 2H), 3.81 (m, 2H), 4.40 (m, 1H), 4.65 (bt, 1H), 6.33 (d, 1H), 7.38 (t, 1H), 7.62 (m, 2H), 7.80 (m, 2H), 8.00 (s, 1H), 8.86 (s, 1H), 9.55 (bs, 1H); ESI, 486 └M+H┘.

### 4-(4-((2,4-dichlorophenethyl)carbamoyl)-2-isobutyramidophenyl)-N-ethyl-1,4-diazepane-1-carboxamide

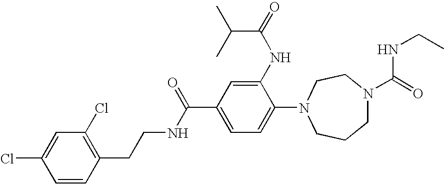

(δ_{II}, 300 MHz, CDCl₃) 1.24 (t, 3H), 1.38 (d, 6H), 2.12 (m 2H), 2.80 (m, 1H), 3.18 (m, 6H), 3.42 (dq, 2H), 3.64 (m, 2H), 3.78 (m, 4H), 4.56 (bt, 1H), 6.48 (d, 1H), 7.25 (m, 3H), 7.50 (d, 1H), 7.77 (dd, 1H), 8.68 (bs, 1H), 8.82 (d, 1H); ESI, 486 [M+H].

### 4-(2-benzamido-4-(4-methylphenethylcarbamoyl)phenyl)-N-ethyl-1,4-diazepane-4-carboxamide

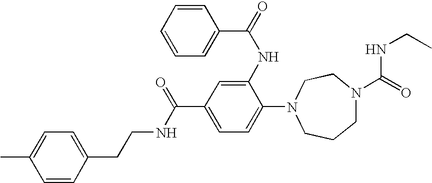

(δ_{H}, 300 MHz, CDCl₃) 1.22 (t, 3H), 2.12 (m, 2H), 2.42 (s, 3H), 3.00 (t, 2H), 3.18 (m, 2H), 3.23 (m, 2H), 3.40 (dq, 2H), 3.70 (t, 2H), 3.78 (m, 4H), 4.58 (bt, 1H), 6.52 (bt, 1H), 7.22 (s 4H), 7.39 (m, 2H), 7.70 (m, 4H), 8.02 (dd, 1H), 8.94 (d, 1H), 9.52 (bs, 1H); ESI, 528 [M+H].

### 4-(4-((2,4-dichlorophenethyl)carbamoyl)-2-(3-fluorobenzamido)phenyl)-N-ethyl-1,4-diazepane-1-carboxamide

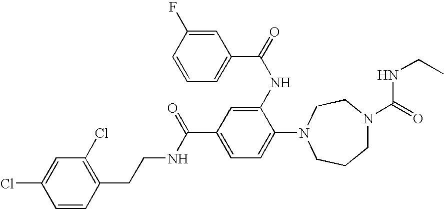

(δ_{H}, 300 MHz, CDCl₃) 1.22 (t 3H), 2.10 (m, 2H), 3.18 (m, 4H), 3.22 (m, 2H), 3.38 (dq, 2H), 3.68 (t, 2H), 3.78 (m, 4H), 4.58 (bt, 1H), 6.61 (bt, 1H), 7.38 (m, 3H), 7.46 (d, 2H), 7.61 (m, 2H), 7.74 (m, 3H), 8.86 (d, 1H), 9.48, (bs, 1H); ESI, 600 [M+H].

### 1-(2-(4-(ethylcarbamoyl)-1,4-diazepan-1-yl)-5-(isopropylcarbamoyl)phenyl)-3-phenylurea

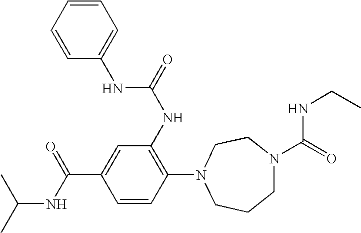

(δ_{H}, 300 MHz, CDCl₃) 1.27 (t, 3H), 1.36 (d, 6H) 1.98 (m, 2H), 3.15 (m, 2H), 3.36 (m, 2H), 3.42 (m, 2H), 3.59 (m, 2H), 3.80 (m, 2H), 4.38 (m, 1H), 5.09 (t, 1H), 6.43 (d, 1H), 7.18 (m, 2H), 7.40 (m, 2H), 7.75 (d, 2H), 8.10 (s, 1H), 8.80 (s, 1H), 9.60 (bs, 1H); ESI 467 [M+H].

### 4-(2-(3-chlorobenzamido)-4-((2,3-dihydro-1H-1-inden-1-yl)carbamoyl)phenyl)-N-ethyl-1,4-diazepane-1-carboxamide

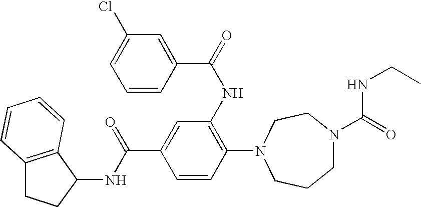

(δ_{H}, 300 M CDCl₃) 1.26 (t, 3H), 2.10 (r, 3H), 2.83 (m, 1H), 3.06 (m, 1H), 3.20 (m, 3H), 3.19 (m, 2H), 3.29 (m, 2H), 3.41 (dq, 2H), 3.72 (t, 2H), 3.81 (m, 2H), 4.46 (t, 1H), 5.84 (q, 1H), 6.63 (d, 1H), 7.35 (m, 4H), 7.47 (m, 1H), 7.63 (m, 2H), 7.85 (m, 2H), 8.02 (m, 1H), 8.92 (d, 1H), 9.50 (bs, 1H); ESL 560 [M+H].

### 4-(4-((2-fluorophenethyl)carbamoyl)-2-(3-chlorobenzamido)phenyl)-N-ethyl-1,4-diazepane-1-carboxamide

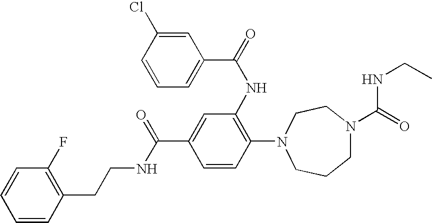

(δ_{H}, 300 MHz CDCl₃) 1.25 (t, 3H), 2.13 (m, 2H), 3.12 (t, 1H), 3.03 (m, 2H), 3.19 (m, 2H), 3.28 (m, 2H), 3.40 (dq, 2H), 3.72 (t, 2H), 3.80 (m, 4H), 4.47 (t, 1H) 6.53 (bt, 1H), 7.20 (m, 2H), 7.38 (m, 3H), 7.65 (m, 2H), 7.78 (dd, 1H), 7.86 (dt, 1H), 8.04 (m, 1H), 8.90 (d, 1H), 9.51 (bs, 1H); ESI, 566 [M+H].

### 4-(4-((4-fluorophenethyl)carbamoyl)-2-(3-chlorobenzamido)phenyl)-N-ethyl-1,4-diazepane-1-carboxamide

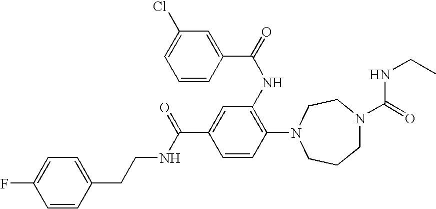

(δ_{H}, 300 MHz, CDCl₃) 1.26 (t, 3H), 2.15 (m, 2H), 3.03 (m, 2H), 3.319 (m, 2H), 3.28 (m, 2H), 3.41 (dq, 2H), 3.72 (t, 2H), 3.80 (m, 4H), 4.46 (t, 1H) 6.48 (bt, 1H), 7.14 (t, 2H), 7.35 (m, 3H), 7.64 (m, 2H), 7.78 (dd, 1H), 7.86 (dt, 1H), 8.03 (m, 1H), 8.89 (d, 1H), 9.51 (bs, 1H); ESL 566 [M+H].

### 4-(4-((2,4-dichlorophenethyl)carbamoyl)-2-(3-chlorobenzamido)phenyl)-N-propyl-1,4-diazepane-1-carboxamide

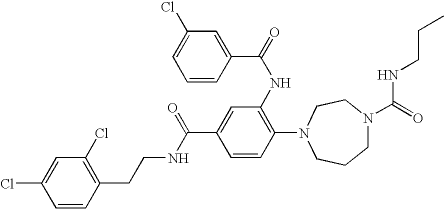

(δ_{H}, 300 MHz, CDCl₃) 1.02 (t, 3H), 1.62 (q, 2H), 2.12 (m, 2H), 3.18 (m, 4H), 3.26 (m, 4H), 3.71 (m, 2H), 3.80 (m, 4H), 4.57 (bt, 1H), 6.52 (bt, 1H), 7.35 (m, 3H), 7.52 (s, 1H), 7.61 (m, 2H), 7.78 (m, 1H), 8.82 (m, 1H), 8.03 (m, 1H), 8.92 (d, 1H), 9.53 (bs, 1H); ESI, 630 [M+H].

### 4-(4-(((+/−)-trans-2-phenylcyclopropyl)carbamoyl)-2-(3-chlorobenzamido)phenyl)-N-ethyl-1,4-diazepane-1-carboxamide

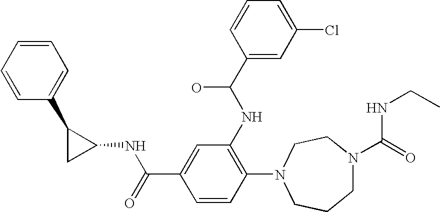

(δ_{H}, 300 MHz, CDCl₃) 1.26 (t, 3H), 1.43 (m, 3H), 2.15 (m, 2H), 2.32 (m, 1H), 3.20 (m, 2H), 3.28 (m, 2H), 3.41 (dq, 2H), 3.72 (t, 2H), 3.82 (m, 2H), 4.46 (t, 1H), 6.75 (bs, 1H), 7.35 (m, 6H), 7.65 (m, 2H), 7.87 (m, 2H), 8.04 (m, 1H), 8.90 (d, 1H), 9.54 (bs, 1H); ESL 560 [M+H].

### 4-(4-((3,4-dimethoxyphenethyl)carbamoyl)-2-(3-chlorobenzamido)phenyl)-N-ethyl-1,4-diazepane-1-carboxamide

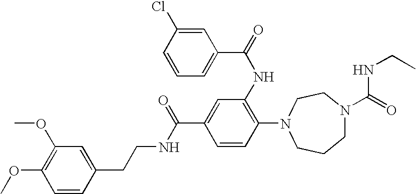

(δ_{H}, 300 MHz, CDCl₃) 1.25 (t, 3H), 2.15 (m, 2H), 3.03 (m, 2H), 3.19 (m, 2H), 3.28 (m, 2H), 3.43 (m, 2H), 3.72 (t, 2H), 3.80 (m, 4H), 3.98 (s, 6H), 4.50 (bt, 1H) 6.65 (bt, 1H), 6.95 (m, 3H), 7.30-8.20 (m, 6H), 8.90 (d, 1H) 9.51 (bs, 1H); ESL, 608 [M+H].

### 4-(4-((2,4-dichlorophenethyl)carbamoyl)-2-(3,5-difluorobenzamido)phenyl)-N-ethyl-1,4-diazepane-1-carboxamide

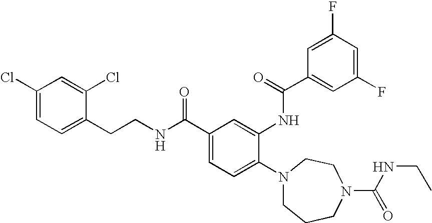

(δ_{H}, 300 MHz, CDCl₃) 1.23 (t, 3H), 2.18 (m, 2H), 3.16 (m, 4H), 3.22 (m, 2H), 3.42 (dq, 2H), 3.71 (t, 2H), 3.80 (m, 4H), 4.60 (bt, 1H) 6.68 (bt, 1H), 7.18 (dt, 3H), 7.38 (m, 3H), 7.52 (m, 3H), 7.79 (dd, 1H), 8.80 (d, 1H), 9.46 (bs, 1H); ESI, 618 [M+H].

### 4-(4-((2,4-dichloropenethyl)carbamoyl)-2-(2-fluorobenzamido)phenyl)-N-ethyl-1,4-diazepane-1-carboxamide

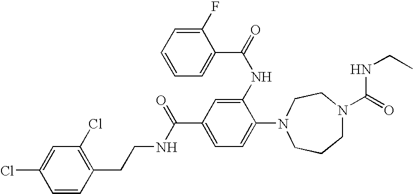

(δ_{H}, 300 MHz, CDCl₃) 1.23 (t, 3H), 2.18 (m, 2H), 3.16 (m, 6H), 3.40 (dq, 2H), 3.71 (t, 2H), 3.80 (m, 4H), 4.60 (bt, 1H) 6.64 (bt, 1H), 7.30 (m, 4H), 7.42 (m, 2H), 7.63 (m, 1H), 7.76 (dd, 1H), 8.35 (dt, 1H), 9.00 (d, 1H); ESI, 600 [M+H].

### 4-(4-((2,4-dichlorophenethyl)carbamoyl)-2-(3-chlorobenzamido)phenyl)-1,4-diazepane

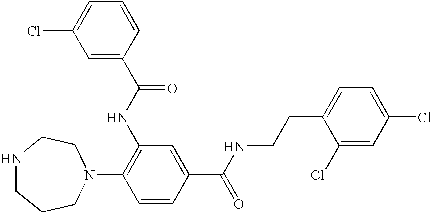

(δ_{H}, 300 MHz CDCl₃) 1.83 (m, 2H), 3.05 (m, 8H), 3.20 (t, 2H), 3.67 (q, 2H), 6.34 (bt, 1H), 7.22 (m, 3H), 7.36 (d, 1H), 7.45 (d, 1H), 7.52 (m, 1H), 7.65 (dd, 1H), 7.85 (m, 1H), 8.03 (m, 1H), 8.73 (m, 1H), 9.82 (bs, 1H); ESI, 545 [M+H].

### 4-(4-((2,4-dichlorophenethyl)carbamoyl)-2-(3-chlorobenzamido)phenyl)-N-ethyl-1,4-diazepane-1-carboxamide

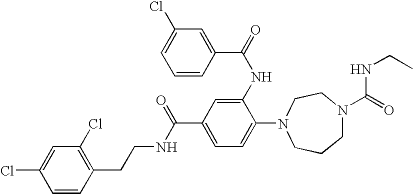

(δ_{H}, 300 MHz, CDCl₃) 1.12 (t, 3H), 2.00 (m, 2H), 3.05 (m, 4H), 3.13 (m, 2H), 3.26 (m, 2H), 3.58 (t, 2H), 3.67 (m, 4H), 4.33 (t, 1H), 6.32 (t, 1H), 7.20 (m, 2H), 7.25 (m, 1H) 7.38 (d, 1H), 7.48 (m, 1H), 7.54 (m, 1H), 7.64 (dd, 1H), 7.73 (m, 1H), 7.90 (m, 1H), 8.77 (d, 1H), 9.38 (bs, 1H); ESI, 616 [M+H].

### (+/−)-4-(2-(3-chlorobenzamido)-4-((1,2,3,4-tetrahydronaphthalen-1-yl)carbamoyl)phenyl)-N-ethyl-1,4-diazepane-1-carboxamide

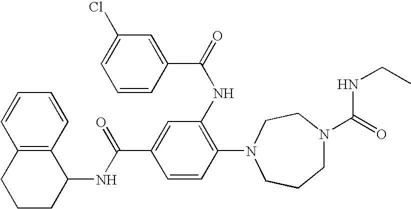

(δ_{H}, 300 MHz, CDCl₃) 1.26 (t, 3H), 2.10 (m, 6H), 2.95 (m, 2H), 3.18 (m, 2H), 3.27 (m, 2H), 3.40 (dq, 2H), 3.71 (t, 2H), 3.82 (m, 2H), 4.45 (t, 1H), 5.03 (m, 1H) 6.64 (d, 1H), 7.25 (m, 3H), 7.42 (m, 2H) 7.62 (m, 2H), 7.85 (m, 2H), 8.01 (t, 1H), 8.90 (d, 1H), 9.49 (bs, 1H); ESI, 574 [M+H].

### 4-(4-(((+/−)(-trans-)-2-phenylcyclopropyl)carbamoyl)-2-benzamidophenyl)-N-ethyl-1,4-diazepane-1-carboxamide

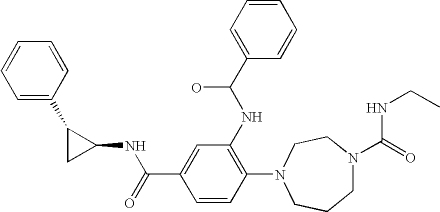

(δ_{H}, 300 MHz, CDCl₃) 1.23 (t, 3H), 1.40 (m, 3H), 2.10 (m, 2H), 2.30 (m, 1H), 3.18 (m, 2H), 3.24 (m, 2H), 3.40 (dq, 2H), 3.74 (t, 2H), 3.80 (m, 2H), 4.53 (t, 1H), 6.80 (d, 1H), 7.35 (m, 6H), 7.65 (m, 3H), 7.82 (dd, 1H), 8.02 (d, 2H), 8.96 (d, 1H), 9.54 (bs, 1H); ESI, 526 └M+H┘.

### 4-(4-((2,4-dichlorophenethyl)carbamoyl)-2-(isoxazole-5-carboxamido)phenyl)-N-ethyl-1,4-diazepane-1-carboxamide

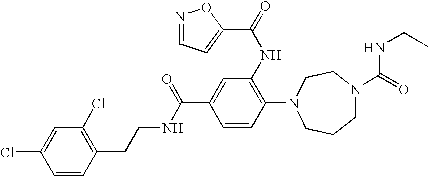

(δ_{II}, 300 MHz CDCl₃) 1.24 (t, 3H), 2.14 (m, 2H), 3.18 (m, 4H), 3.22 (m, 2H), 3.43 (dq, 2H), 3.78 (m, 4H), 3.91 (m, 2H), 4.52 (bt, 1H), 6.42 (bt, 1H), 7.19 (d, 1H), 7.30 (m, 3H), 7.52 (d, 1H), 7.79 (dd, 1H), 8.54 (d, 1H), 7.84 (d, 1H), 9.85 (bs, 1H); ESI, 573 └M+H┘.

### 4-(4-((2,4-dichlorophenethyl)carbamoyl)-2-(thiophene-4-carboxamide)phenyl)-N-ethyl-1,4-diazepane-1-carboxamide

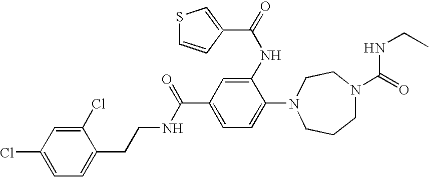

(δ_{H}, 300 MHz, CDCl₃) 1.23 (t 3H), 2.10 (m, 2H), 3.15 (m, 4H), 3.25 (m, 2H), 3.43 (dq, 2H), 3.72 (t, 2H), 3.78 (m, 4H), 4.54 (bt, 1H), 6.58 (bt, 1H), 7.35 (m, 3H), 7.48 (d, 1H), 7.56 (m, 1H), 7.62 (m, 1H), 7.77 (dd, 1H), 8.14 (d, 1H), 8.86 (d, 1H), 9.85 (bs, 1H); ESI, 588 └M+H┘.

### 4-(4-((3-fluorophenethyl)carbamoyl)-2-(3-chlorobenzamido)phenyl)-N-ethyl-1,4-diazepane-1-carboxamide

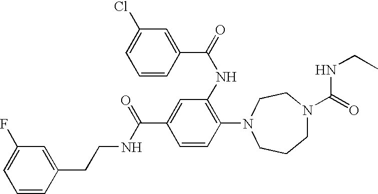

(δ_{II}, 300 MHz, CDCl₃) 1.26 (t, 3H), 2.14 (m, 2H), 3.06 (t, 2H), 3.18 (m, 2H), 3.28 (m, 2H), 3.41 (dq, 2H), 3.72 (m, 2H), 3.81 (m, 4H), 4.46 (t, 1H), 6.50 (t, 1H), 7.05 (r, 2H), 7.16 (d, 1H), 7.40 (m, 2H), 7.62 (m, 2H), 7.78 (dd, 1H), 7.85 (dt, 1H), 8.03 (m, 1H), 8.89 (d, 1H), 9.51 (bs, 1H); ESI, 566 ┌M+H┐.

### 4-(4-((2,4-dichlorophenethyl)carbamoyl)-2-(furan-2-carboxamido)phenyl)-N-ethyl-1,4-diazepine-1-carboxamide

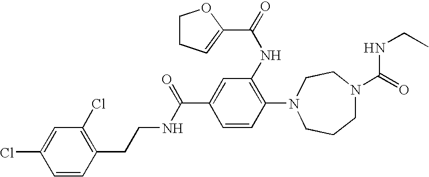

(δ_{H}, 300 MHz, CDCl₃) 1.24 (t. 3H), 2.20 (m, 2H), 3.12 (4H), 3.22 (m, 2H), 3.41 (dq, 2H), 3.80 (m, 6H), 4.59 (bt, 1H), 6.58 (bt, 1H), 6.70 (dd, 1H), 7.38 (m, 4H), 7.46 (d, 1H), 7.67 (d, 1H), 7.75 (dd, 1H), 8.86 (d, 1H), 9.60 (bs, 1H); ESI, 572 └M+H┘.

### 4-(4-((2,4-dichlorophenethyl)carbamoyl-2-(isonicotinamido)phenyl)-N-ethyl-1,4-diazepan-1-carboxamide

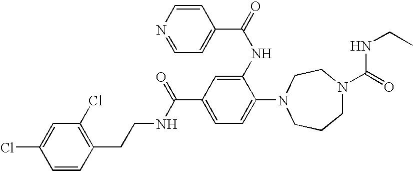

(δ_{II}, 300 MHz, CDCl₃) 1.22 (t, 3H), 2.10 (m, 2H), 3.19 (m, 4H), 3.28 (m, 2H), 3.38 (dq, 2H), 3.71 (t, 2H), 3.79 (m, 4H), 4.43 (bt, 1H), 6.43 (bt, 1H), 7.38 (m, 3H), 7.51 (d, 1H), 7.80 (d, 1H), 7.83 (d, 2H), 8.92 (d, 1H); 8.99 (d, 2H), 9.63 (bs, 1H); ESL 583 [M+H].

### 4-(4-((2,4-dichlorophenethyl)carbamoyl)-2-(3-chlorobenzamido)phenyl)-N-butyl-1,4-diazepane-1-carboxamide

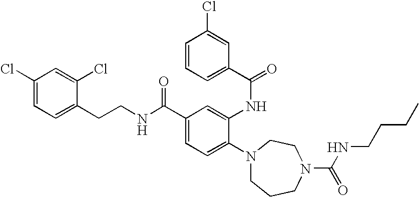

(δ_{H}, 300 MHz, CDCl₃) 1.02 (t, 3H), 1.43 (m, 2H), 1.62 (m, 2H), 2.12 (m, 2H), 3.18 (m, 4H), 3.24 (m, 2H), 3.37 (m, 2H), 3.71 (t, 2H), 3.79 (m, 4H), 4.51 (bt, 1H), 6.53 (bt, 1H), 7.38 (m, 3H), 7.50 (d, 1H), 7.78 (dd, 1H), 7.82 (d, 2H), 8.02 (d, 1H), 8.84 (d, 2H), 9.53 (bs, 1H); ESL, 644 [M+H].

### 4-(2-(3-chlorobenzamido)-4-(phenethylcarbamoyl)phenyl)-N-ethyl-1,4-diazepane-1-carboxamide

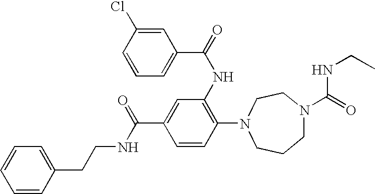

(δ_{H}, 300 MHz, CDCl₃) 1.24 (t, 3H), 2.12 (m, 2H), 3.04 (t, 2H), 3.18 (m, 2H), 3.24 (m, 2H), 3.39 (dq, 2H), 3.71 (t, 2H), 3.80 (m, 4H), 4.54 (bt, 1H), 6.54 (bt, 1H), 7.40 (m, 6H), 7.62 (m, 2H) 7.77 (dd, 1H), 7.84 (dt, 2H), 8.02 (d, 1H), 8.86 (d, 2H), 9.52 (bs, 1H); ESI, 548 [M+H].

### 4-(4-((2,4-dichlorophenethyl)carbamoyl)-2-(3,4-difluorobenzamido)phenyl)-N-ethyl-1,4-diazepane-1-carboxamide

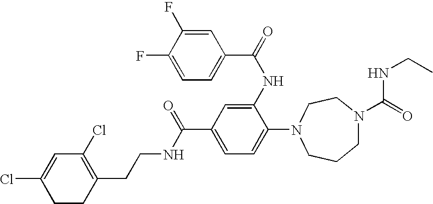

(⊕_{H}, 300 MHz, CDCl₃) 1.22 (t, 3H), 2.10 (m, 2H), 3.14 (m, 4H), 3.24 (m, 2H), 3.39 (dq, 2H), 3.70 (t, 2H), 3.78 (m, 4H), 4.57 (bt, 1H), 6.56 (bt, 1H), 7.35 (m, 3H), 7.44 (m, 2H), 7.72 (m, 2H), 7.91 (dt, 2H), 8.82 (d, 1H), 9.44 (bs, 1H); ESI, 618 [M+H].

### 4-(4-((4-methylphenethyl)carbamoyl)-2-(3-chlorobenzamido)phenyl)-N-ethyl-1,4-diazepane-1-carboxamide

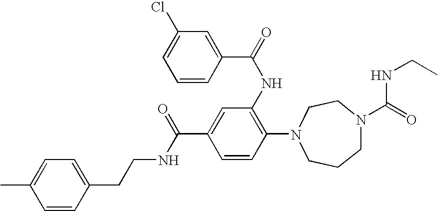

(δ_{H}, 300 MHz, CDCl₃) 1.27 (t, 3H), 2.13 (m, 2H), 2.45 (s, 3H), 3.02 (t, 2H), 3.18 (m, 2H), 3.27 (m, 2H), 3.41 (dq, 2H), 3.72 (m, 2H), 3.81 (m, 4H), 4.44 (t, 1H), 6.40 (t, 1H), 7.39 (m, 4H), 7.65 (m, 2H), 7.78 (dd, 1H), 7.87 (dt, 1H), 8.05 (m, 1H), 8.90 (d, 1H), 9.51 (bs, 1H); ESL, 562 [M+H].

### 4-(2-(3-chlorobenzamido)-4-((2,3-dihydro-1H-inden-2-yl)carbamoyl)phenyl)-N-ethyl-1,4-diazepane-1-carboxamide

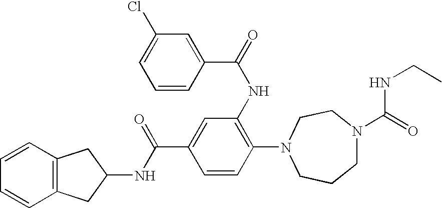

(δ_{H}, 300 MHz, CDCl₃) 1.25 (t, 3H), 2.12 (m, 2H), 3.08 (dd, 2H), 3.17 (m, 2H), 3.26 (m, 2H), 3.40 (dq, 2H), 3.55 (dd, 2H), 3.71 (t, 2H), 3.82 (m, 2H), 4.45 (t, 1H), 5.06 (m, 1H), 6.72 (d, 1H), 7.35 (m, 3H), 7.62 (m, 3H), 7.82 (m, 3H), 8.03 (m, 1H), 8.84 (d, 1H), 9.49 (bs, 1H); ESI, 574 [M+H].

### 4-(4-((+/−)(trans-2-phenylcyclopropyl)carbamoyl)-2-(isonicotinamido)phenyl)-N-ethyl-1,4-diazepane-1-carboxamide

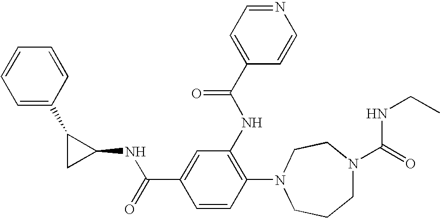

(δ_{H}, 300 MHz, CDCl₃) 1.23 (t, 3H), 1.40 (m, 3H), 2.10 (m, 2H), 2.30 (m, 1H), 3.20 (m, 2H), 3.26 (m, 2H), 3.38 (dq, 2H), 3.70 (t, 2H), 3.30 (m, 2H), 4.48 (t, 1H), 6.80 (d, 1H), 7.35 (m, 6H), 7.84 (m, 3H), 8.90 (d, 1H), 8.98 (d, 2H), 9.63 (bs, 1H); ESI 527 [M+H].

### 4-(2-(3-chlorobenzamido)-4-((2-(thiophen-2-yl)ethyl)carbamoyl)phenyl-N-ethyl-1,4-diazepane-1-carboxamide

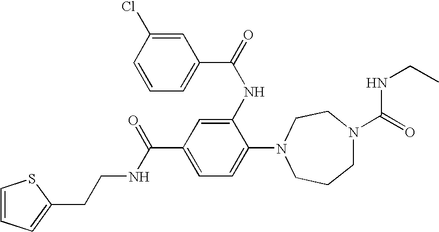

(δ_{H}, 300 MHz, CDCl₃) 1.22 (t 3H), 2.14 (m, 2H), 3.19 (m, 2H), 3.23 (m, 2H), 3.38 (dq, 2H), 3.72 (t, 2H), 3.81 (m, 4H), 4.44 (t, 1H), 6.52 (t, 1H), 7.01 (d, 1H), 7.07 (dd, 1H), 7.28 (dd, 1H), 7.38 (m, 1H), 7.60 (m, 2H), 7.78 (dd, 1H), 7.84 (dd, 1H), 8.02 (d, 1H), 8.92 (d, 1H), 9.52 (bs, 1H); ESL 554 [M+H].

### 4-(2-(3-chlorobenzamido)-4-(isoquinolin-5-ylcarbamoyl)phenyl)-N-ethyl-1,4-diazepane-1-carboxamide

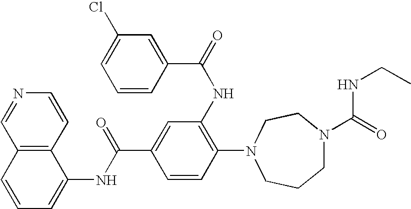

(δ_{H}, 300 MHz, CDCl₃) 1.25 (t, 3H), 2.18 (m, 2H), 3.24 (m, 2H), 3.32 (m, 2H), 3.41 (m, 2H), 3.74 (t, 2H), 3.85 (m, 2H), 4.48 (t, 1H), 7.48 (d, 1H), 7.65 (m, 2H), 7.78 (t, 1H), 7.87 (m, 2H), 7.99 (m, 2H), 8.08 (m, 1H), 8.34 (d, 1H), 8.71 (m, 2H), 9.26 (d, 1H), 9.41 (s, 1H), 9.55 (bs, 1H); ESI, 571 [M+H].

### 4-(4-((2,4-dichlorophenethyl)carbamoyl)-2-benzamidophenyl)-N-ethyl-1,4-diazepane-1-carboxamide

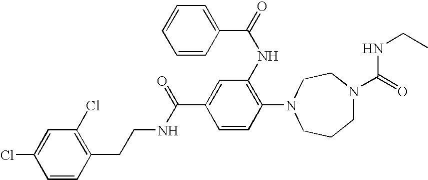

(δ_{H}, 300 MHz, CDCl₃) 1.10 (t, 3H), 2.00 (m, 2H), 3.05 (m, 4H), 3.15 (m, 2H), 3.28 (dq, 2H), 3.59 (t, 2H), 3.66 (m, 4H), 4.28 (t, 1H), 6.32 (t, 1H), 7.25 (m, 3H), 7.38 (d, 1H), 7.54 (m, 3H), 7.65 (dd, 1H), 7.89 (m, 2H), 8.82 (d, 1H), 9.40 (bs, 1H); ESL 582 [M+H].

The functional antagonists of the chemokine receptor CXCR3 disclosed above were identified based on the inhibition of both calcium mobilization and T-cell chemotaxis in response to stimulation with I-TAC. In addition, the compounds were shown to be non-cytotoxic.

### CXCR3 FLIPR® Assays:

ACXCR3 cDNA clone, (sequence as listed in Genbank, accession number BD 195161) and chimeric G protein Gqi5, were used to construct a stably transfected HEK293 cell line using co-transfection protocols known to those of skill in the art. HEK293/CXCR3 G_{qi5} cells were seeded at 10,000 cells (25 μL) per well in poly (D-lysine)-treated 384-well plates (Costar, black clear-bottom cell culture-treated) 24-48 hours prior to the assay. Culture medium was removed and replaced with 25 μL of 50% cell culture medium/50% Calcium Plus Dye (Molecular Devices)/2.5 mM probenecid (Sigma). For dye loading, plates were incubated for 30 minutes at 37° C./5% CO₂, followed by equilibration to room temperature for 30-90 min. Test compounds were diluted in 20 μL HBSS/20 mM HEPES, pH7.5/1% DMSO/0.1% BSA/2.5 probenecid. 12.5 μL test compound (or as controls, CXCL11/I-TAC to 40 nM or buffer alone, also with 1% DMSO) was added in the FLIPR® 384 to dye-loaded cells. 12.5 μL ITAC (R&D Systems), in HBSS/20 mM HEPES, pH 7.5/0.1% BSA, was then added to the cells/test compound, to a final concentration of 40 nM, and fluorescence measured once per second over the first minute, followed by an additional two minutes of one measurement/two seconds. All FLIPR® pipette tips were presoaked in 1% BSA prior to use in order to reduce adsorption of ligand.

### CXCR3 Radioligand Binding Assay:

(¹²⁵I) CXCL10/IP-10 (NEN) at 25 nM was allowed to bind at 25° C. to crude HEK293/CXCR3 Gqi5 membrane preparations in 50 mM HEPES, pH 7.5, 5 mM MgCl₂, 1 mM CaCl₂, 0.5% BSA, 1% DMSO in the presence of test compounds. Reactions were filtered through 0.3% polyethyleneimine-blocked MAFCNOB filter plates (Millipore) and washed three times with ice-cold 50 mM HEPES, pH 7.5, 0.5 M NaCl, 0.1% BSA. 1 μM unlabeled CXCL9/Mig (Peprotech) was used to define nonspecific binding.

### Cytotoxicity Assay

20,000 HEK293/CXCR3 Gqi5 cells were seeded in clear 96-well tissue culture-treated plates in 50 μL, in culture medium without DMSO. 50 μL of the test compounds, (serially diluted in medium/2% DMSO) or Triton X-100/2% DMSO as a control were added, followed by incubation for 24 hours at 37° C./CO₂. 10 uL WST-1 reagent (Roche) were added and plates incubated at 37° C. until color developed. After agitation of the plates for 5 minutes, absorbance at 450 nm was measured.

### Formulations

While it may be possible for the compounds of the present invention to be administered as the raw chemical, it is preferable to present them as a pharmaceutical composition. According to a further aspect, the present invention provides a pharmaceutical composition comprising a compound of formula I or a pharmaceutically acceptable salt or solvate thereof, together with one or more pharmaceutically acceptable carriers thereof and optionally one or more other therapeutic ingredients, as discussed below. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the Recipient. The formulations may conveniently be presented in unit dosage form and may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided, solid carriers or both and then, if necessary, shaping the product into the desired formulation.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids. Including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. A tablet may be made by compression or molding, optionally with one or more accessory ingredients. Compressed tablets may be prepared by compressing in a suitable machine the active ingredient in a free-flowing form such as a powder or granules, optionally mixed with a hinder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia. It should be understood that in addition to the ingredients particularly mentioned above, the formulations of this invention may include other agents conventional in the art having regard to the type of formulation in question, for example those suitable for oral administration may include flavoring agents.

Preferred unit dosage formulations are those containing an effective dose, as recited below, or an appropriate fraction thereof, of the active ingredient. The compounds of the invention may be administered orally or via injection at a dose from 0.001 to 2500 mg/kg per day. The dose range for adult humans is generally from 0.005 mg to 10 g/day. Tablets of other forms of presentation provided in discrete units may conveniently contain an amount of compound of the invention which is effective at such dosage or as a multiplex of the same, for instance, units containing 5 mg to 500 mg, usually around 10 mg to 200 mg.

The compounds of formula (I) are preferably administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

1. A compound of formula I 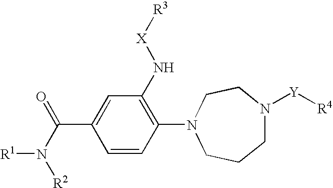
wherein:
R¹ is substituted or unsubstituted alkyl substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;
R² is H;
X is CO—, or (CO)—NH—;
R³ is substituted or unsubstituted C2-C6 alkyl substituted or unsubstituted cycloalkyl, substituted or unsubstituted aryl, substituted or unsubstituted heteroaryl, substituted or unsubstituted heterocycle, substituted or unsubstituted arylalkyl, substituted or unsubstituted heteroarylalkyl;
Y is H, C(O)—, CON—, or C(O)NH—; and
R⁴ is H, or substituted or unsubstituted alkyl
wherein R³ is not pyridine when R¹ is alkyl.

2. A 1,4-diazepane according to claim 1 of formula I 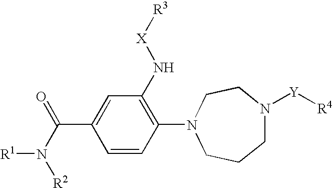
wherein
X is CO, Y is CONH—, and R² is H.

3. A 1,4-diazepane carboxamide according to claim 2, of formula II 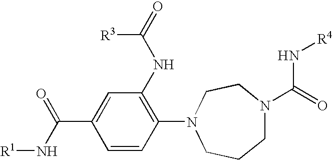
wherein
R⁴ is alkyl.

4. A compound according to claim 3 of formula II: 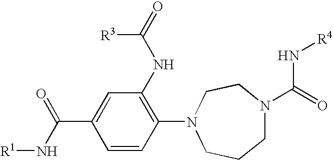
wherein
R¹ is substituted or unsubstituted alkyl substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl; and
R³ is substituted or unsubstituted C2-C6 alkyl, substituted or unsubstituted cycloalkyl, substituted or unsubstituted aryl, substituted or unsubstituted heteroaryl, substituted or unsubstituted heterocycle; substituted or unsubstituted arylalkyl, substituted or unsubstituted heteroarylalkyl;
wherein R³ is not pyridine when R¹ is alkyl.

5. A compound according to claim 4 of formula III 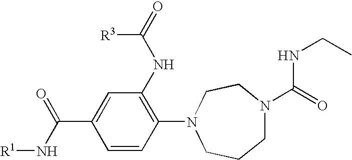
wherein
R¹ is substituted or unsubstituted alkyl, substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;
R³ is substituted or unsubstituted aryl or substituted or unsubstituted heteroaryl;
wherein R³ is not pyridine when R¹ is alkyl.

6. A compound according to claim 4 of formula III 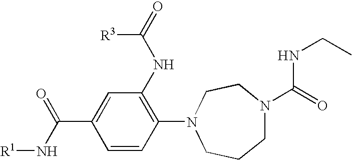
wherein R¹ is substituted or unsubstituted arylalkyl; and
R³ is substituted or unsubstituted aryl or heteroaryl.

7. A compound according to claim 1 chosen from:
4-(4-((4-fluorophenethyl)carbamoyl)-2-benzamidophenyl)-N-ethyl-1,4-diazepane-1-carboxamide;
4-(4-((2,4-dichlorophenethyl)carbamoyl)-2-(furan-4-carboxamido)phenyl)-N-ethyl-1,4-diazepane-1,7-carboxamide;
4-(4-((2,4-dichlorophenethyl)carbamoyl)-2-cyclopropanecarboxamido)phenyl)-N-ethyl-1,4-diazepane-1-carboxamide;
(+/−)-4-(2-(3-chlorobenzamido)-4-((2-phenylpropyl)carbamoyl)phenyl)-N-ethyl-1,4-diazepane-1-carboxamide;
4-(2-(3-chlorobenzamido)-4-(isopropylcarbamoyl)phenyl)-acetyl-1,4-diazepane;
4-(4-(((+/−)trans-2-phenylcyclopropyl)carbamoyl)-2-(3,5-difluorobenzamido)phenyl)-N-ethyl-1,4-diazepane-1-carboxamide;
4-(2-(3-chlorobenzamido)-4-((3-phenylpropyl)carbamoyl)phenyl)-N-ethyl-1,4-diazepane-1-carboxamide;
4-(4-((4-chlorobenzyl)carbamoyl)-2-(3-chlorobenzamido)phenyl)-N-ethyl-1,4-diazepane-1-carboxamide;
4-(4-((4-chlorophenethyl)carbamoyl)-2-benzamidophenyl)-N-ethyl-1,4-diazepane-1-carboxamide;
4-(2-(3-chlorobenzamido)-4-((naphthalen-1-ylmethyl)-carbamoyl)phenyl)-N-ethyl-1,4-diazepane-1-carboxamide;
4-(4-((2,4-dichlorophenethyl)carbamoyl)-2-(3-chlorobenzamido)phenyl)-N-isopropyl-1,4-diazepane-1-carboxamide;
4-(4-((2,4-dichlorophenethyl)carbamoyl)-2-(4-chlorobenzamido)phenyl)-N-ethyl-1,4-diazepane-1-carboxamide;
4-(4-((4-chlorophenethyl)carbamoyl)-2-(3-chlorobenzamido)phenyl)-N-ethyl-1,4-diazepane-1-carboxamide;
4-(4-((4-chlorophenethyl)carbamoyl)-2-(3-chlorobenzamido)phenyl)-N-ethyl-1,4-diazepane-1-carboxamide;
4-(4-(2,4-dichlorophenethyl)carbamoyl)-2-(3-methoxybenzamido)phenyl)-N-ethyl-1,4-diazepane-1-carboxamide;
4-(4-(benzylcarbamoyl-2-(3-chlorobenzamido)phenyl)-N-ethyl-1,4-diazepane-1-carboxamide;
4-(4-((2,4-dichlorophenethyl)carbamoyl)-2-(2-chlorobenzamido)phenyl)-N-ethyl-1,4-diazepane-1-carboxamide;
4-(4-((2,4-dichlorophenethyl)carbamoyl)-2-(thiophene-2-carboxamido)phenyl)-N-ethyl-1,4-diazepane-1-carboxamide;
4-(4-((2,4-dichlorophenethyl)carbamoyl)-2-(3-chlorobenzamido)phenyl)-N-methyl-1,4-diazepane-1-carboxamide;
4-(2-benzamido-4-(3-phenylpropyl)carbamoyl)phenyl-ethyl-1,4-diazepane-1-carboxamide;
4-(2-(3-chlorobenzamido)-4-(3,4-dimethoxyphenethylcarbamoyl)phenyl)-acetyl-1,4-diazepane;
4-(4-(2,4-dichlorophenethyl)carbamoyl)-2-(3-cyanobenzamido)phenyl)-N-ethyl-1,4-diazepane-1-carboxamide;
4-(4-((3-chlorophenethyl)carbamoyl)-2-(3-chlorobenzamido)phenyl)-N-ethyl-1,4-diazepane-1-carboxamide;
4-(4-((4-chloro-2-methylphenethyl)carbamoyl)-2-(4-fluorobenzamido)phenyl)-N-ethyl-1,4-diazepane-1-carboxamide;
4-(2-(3-chlorobenzamido)-4-(isopropylcarbamoyl)phenyl)-N-ethyl-1,4-diazepane-1-carboxamide;
4-(4-((2,4-dichlorophenethyl)carbamoyl)-2-isobutyramidophenyl)-N-ethyl-1,4-diazepane-1-carboxamide;
4-(2-benzamido-4-(4-methylphenethylcarbamoyl)phenyl)-N-ethyl-1,4-diazepane-1-carboxamide;
4-(4-((2,4-dichlorophenethyl)carbamoyl)-2-(3-fluorobenzamido)phenyl)-N-ethyl-1,4-diazepane-1-carboxamide;
1-(2-(4-(ethylcarbamoyl)-1,4-diazepan-1-yl)-5-(isopropylcarbamoyl)phenyl)-3-phenylurea,
4-(2-(3-chlorobenzamido)-4-((2,3-dihydro-1H-inden-1-yl)carbamoyl)phenyl)-N-ethyl-1,4-diazepane-1-carboxamide;
4-(4-((2-fluorophenethyl)carbamoyl)-2-(3-chlorobenzamido)phenyl)-N-ethyl-1,4-diazepane-1-carboxamide;
4-(4-((4-dichlorophenethyl)carbamoyl)-2-(3-chlorobenzamido)phenyl-N-ethyl-1,4-diazepane-1-carboxamide;
4-(4-((2,4-dichlorophenethyl)carbamoyl)-2-(3-chlorobenzamido)phenyl)-N-propyl-1,4-diazepane-1-carboxamide;
4-(4-(((+/−)-trans-2-phenylcyclopropyl)carbamoyl)-2-(3-chlorobenzamido)phenyl)-N-ethyl-1,4-diazepane-1-carboxamide;
4-(4-((3,4-dimethoxyphenethyl)carbamoyl)-2-(3-chlorobenzamido)phenyl)-N-ethyl-1,4-diazepane-1-carboxamide,
4-(4-((2,4-dichlorophenethyl)carbamoyl)-2-(3,5-difluorobenzamido)phenyl)-N-ethyl-1,4-diazepane-1-carboxamide;
4-(4-((2,4-dichlorophenethyl)carbamoyl-2-(2-fluorobenzamido)phenyl)-N-ethyl-1,4-diazepane-1-carboxamide;
4-(4-((2,4-dichlorophenethyl)carbamoyl)-2-(3-chlorobenzamido)phenyl)-1,4-diazepane;
4-(4-((2,4-dichlorophenethyl)carbamoyl)-2-(3-chlorobenzamido)phenyl)-N-ethyl-1,4-diazepane-1-carboxamide;
(+/−)-4-(2-(3-chlorobenzamido)-4-((1,2,3,4-tetrahydronaphthalen-1-yl)carbamoyl)phenyl)-N-ethyl-1,4-diazepane-1-carboxamide;
4-(4-(((+/−)(-trans-)-2-phenylcyclopropyl)carbamoyl)-2-benzamidophenyl)-N-ethyl-1,4-diazepane-1-carboxamide;
4-(4-((2,4-dichlorophenethyl)carbamoyl)-2-(thiophene-4-carboxamido)phenyl)-N-ethyl-1,4-diazepane-1-carboxamide;
4-(4-((2,4-dichlorophenethyl)carbamoyl)-2-(thiophene-4-carboxamido)phenyl)-N-ethyl-1,4-diazepane-1-carboxamide;
4-(4-((3-fluorophenethyl)carbamoyl)-2-(3-chlorobenzamido)phenyl)-N-ethyl-1,4-diazepane-1-carboxamide;
4-(4-((2,4-dichlorophenethyl)carbamoyl)-2-(furan-2-carboxamido)phenyl)-N-ethyl-1,4-diazepane-1-carboxamide;
4-(4-((2,4-dichlorophenethyl)carbamoyl)-2-(isonicotinamido phenyl)-N-ethyl-1,4-diazepane-1-carboxamide;
4-(4-((2,4-dichlorophenethyl)carbamoyl)-2-(3-chlorobenzamido)phenyl)-N-butyl-1,4-diazepane-1-carboxamide;
4-(2-(3-chlorobenzamido)-4-(phenethylcarbamoyl)phenyl)-N-ethyl-1,4-diazepane-1-carboxamide;
4-(4-((2,4-dichlorophenethyl)carbamoyl)-2-(3,4-difluorobenzamido)phenyl)-N-ethyl-1,4-diazepane-1-carboxamide;
4-(4-((4-methylphenethyl)carbamoyl)-2-(3-chlorobenzamido)phenyl)-N-ethyl-1,4-diazepane-1-carboxamide;
4-(2-(3-chlorobenzamido)-4-((2,3-dihydro-1H-inden-2-yl)carbamoyl)phenyl)-N-ethyl-1,4-diazepane-1-carboxamide;
4-(4-((+/−)(trans-2-phenylcyclopropyl)carbamoyl)-2-(isonicotinamido)phenyl)-N-ethyl-1,4-diazepane-1-carboxamide;
4-(2-(3-chlorobenzamido)-4-((2-(thio-2-yl)ethyl)carbamoyl)phenyl)-N-ethyl-1,4-diazepane-1-carboxamide;
4-(2-(3-chlorobenzamido)-4-(isoquinolin-5-ylcarbamoyl)phenyl)-N-ethyl-1,4-diazepane-1-carboxamide;
4-(4-((2,4-dichlorophenethyl)carbamoyl)-2-benzamidophenyl)-ethyl-1,4-diazepane-1-carboxamide

8. A pharmaceutical composition comprising a pharmaceutically acceptable carrier and a therapeutically effective amount of at least one compound according to claim 1.

9. A pharmaceutical composition comprising a pharmaceutically acceptable carrier and a therapeutically effective amount of at least one compound according to claim 7.

10. A method for treating a disorder mediated by CXCR3 function comprising administering to a subject in need of such treatment a therapeutically effective amount of a compound of formula I. 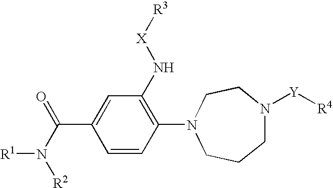
wherein:
R¹ is substituted or unsubstituted alkyl, substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;
R² is H;
X is CO—, or (CO)—NH—;
R³ is substituted or unsubstituted C2-C6 alkyl, substituted or unsubstituted cycloalkyl, substituted or unsubstituted aryl, substituted or unsubstituted heteroaryl, substituted or unsubstituted heterocycle, substituted or unsubstituted arylalkyl, substituted or unsubstituted heteroarylalkyl;
Y is H, C(O)—, CON—, or C(O)NH—; and
R⁴ is H, or substituted or unsubstituted alkyl,
wherein R³ is not pyridine when R¹ is alkyl.

11. The method of claim 10 wherein said compound is a 1,4-diazapene carboxamide of the formula 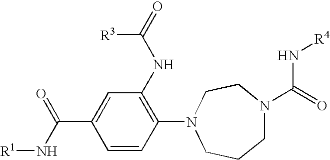
wherein
R¹ is substituted or unsubstituted alkyl, substituted or unsubstituted cycloalkyl, substituted or unsubstituted aryl, substituted or unsubstituted arylcycloalkyl substituted or unsubstituted heteroaryl, substituted or unsubstituted heterocycle, substituted or unsubstituted arylalkyl, substituted or unsubstituted sulfur or oxygen heteroarylalkyl;
R³ is substituted or unsubstituted C2-C6 alkyl, substituted or unsubstituted cycloalkyl, substituted or unsubstituted aryl, substituted or unsubstituted heteroaryl, substituted or unsubstituted heterocycle, substituted or unsubstituted arylalkyl, substituted or unsubstituted heteroarylalkyl;
and
R⁴ is H, or substituted or unsubstituted alkyl,
wherein R³ is not pyridine when R¹ is alkyl.

12. The method of claim 10 wherein said disorder mediated by CXCR3 function is inflammation.

13. The method of claim 10 wherein said disorder is inflammatory bowel disease.

14. The method of claim 10 wherein said disorder is insulitis associated with diabetes.

15. The method of claim 10 wherein said disorder is rheumatoid arthritis.

16. The method of claim 10 wherein said disorder is multiple sclerosis.

17. A method for treating inflammation comprising administering to a mammal a therapeutically effective amount of a compound according to claim 1.
